(19) United States
(12) Patent Application Publication
(10) Pub. No.: US 2026/0069856 A1
(43) Pub. Date: Mar. 12, 2026

(54) Explant Plug Devices for Use in VAD Explantation

(71) Applicant: TC1 LLC, St. Paul, MN (US)

(72) Inventors: Daniel I. Harjes, Carlisle, MA (US); Rachel Keen, Medford, MA (US); Rebecca Ramos, Berkley, MA (US); Stephen Franklin, Haverhill, MA (US); Samantha Cashton, Wantagh, NY (US); Elizabeth Afanasewicz, Allston, MA (US); Grey Nagle, Wilmington, MA (US); Christopher Grace, St. Paul, MN (US)

(73) Assignee: TC1 LLC, St. Paul, MN (US)

(21) Appl. No.: 19/389,771

(22) Filed: Nov. 14, 2025

Related U.S. Application Data
(63) Continuation of application No. PCT/US2024/029926, filed on May 17, 2024.
(60) Provisional application No. 63/467,240, filed on May 17, 2023.
(60) Provisional application No. 63/615,925, filed on Dec. 29, 2023.
(60) Provisional application No. 63/648,562, filed on May 16, 2024.

(51) Int. Cl. A61M 60/863; A61M 60/178

(52) U.S. Cl. CPC A61M 60/863; A61M 60/178

(57) ABSTRACT

Explant plug devices for attachment to a ventricular cuff during explantation of a ventricular assist device. An explant plug device includes a blood flow blocking component and a retention clip. The blood flow blocking component is configured to be mounted to the ventricular cuff to block flow of blood out of the ventricle via the ventricular cuff. The retention clip is coupled with the blood flow blocking component. The retention clip is repositionable relative to the blood flow blocking component from a retracted position that accommodates mounting of the blood flow blocking component to the ventricular cuff and a retention position that retains the blood flow blocking component to the ventricular cuff.

### CROSS REFERENCE TO RELATED APPLICATION DATA

The present application is a Continuation of PCT/US2024/029926 filed May 17, 2024; which claims the benefit of U.S. Provisional Appln. Nos. 63/467,240 filed May 17, 2023, 63/615,925 filed Dec. 29, 2023, and 63/648,562 filed May 16, 2024; the full disclosures which are incorporated herein by reference in their entirety for all purposes.

### BACKGROUND

Ventricular assist devices, known as VADs, are implantable blood pumps used for both short-term (i.e., days, months) and long-term applications (i.e., years or a lifetime) where a patient's heart is incapable of providing adequate circulation, commonly referred to as heart failure or congestive heart failure. According to the American Heart Association, more than five million Americans are living with heart failure, with about 670,000 new cases diagnosed every year. People with heart failure often have shortness of breath and fatigue. Years of living with blocked arteries or high blood pressure can leave a heart too weak to pump enough blood to the body. As symptoms worsen, advanced heart failure develops.

A patient suffering from heart failure, also called congestive heart failure, may use a VAD while awaiting a heart transplant or as a long term destination therapy. In another example, a patient may use a VAD while their own native heart recovers. Thus, a VAD can supplement a weak heart (i.e., partial support) or can effectively replace the natural heart's function. VADs can be implanted in the patient's body and powered by an electrical power source inside or outside the patient's body.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Embodiments disclosed herein are directed to explant plug devices configured to replace a VAD attached to a ventricle via a ventricular cuff to block blood flow out of the ventricle through the ventricular cuff. In many embodiments, the explant plug device occupies less space than the VAD and therefore may provide associated benefits following explantation of the VAD. The explant plug device can be used to avoid having to perform a ventricular patch plasty during explantation of a VAD.

In one aspect, an explant plug device is mountable to a ventricular cuff in fluid communication with a ventricle to block blood from flowing out of the ventricle through a blood flow channel of the ventricular cuff. The explant plug device includes a seal assembly and a retention assembly. The seal assembly is configured for insertion into the blood flow channel and blocking blood flow through the blood flow channel. The retention assembly includes one or more retention members and a spring assembly coupled with the one or more retention members. The retention assembly is reconfigurable between an insertion configuration and a retention configuration. Each of the one or more retention members in the insertion configuration is positioned to accommodate insertion of the retention assembly into the blood flow channel of the ventricular cuff. The spring assembly in the retention configuration is configured to hold each of the one or more retention members in engagement with an inner surface of the ventricular cuff that defines the blood flow channel.

In some embodiments, the spring assembly includes one or more preloaded springs. The preloaded springs can be configured to hold each of the one or more retention members in engagement with the inner surface of the ventricular cuff in the retention configuration. In some embodiments, the one or more retention members include a first retention member and a second retention member. The first retention member can be configured for engaging a first annular portion of the inner surface in the retention configuration. The second retention member can be configured for engaging a second annular portion of the inner surface in the retention configuration. The second annular portion of the inner surface is disposed opposite to the first annular portion of the inner surface.

In some embodiments, the retention assembly includes a frame member to which the seal assembly is attached. Each of the one or more retention members can be coupled with the frame member to accommodate movement of the retention member relative to the frame member.

The seal assembly can have any suitable configuration. For example, the seal assembly can be detachably mounted to the retention assembly. The seal assembly can include an O-ring seal configured to interface with the inner surface of the blood flow channel. The seal assembly can be configured for filling an opening in a ventricular wall to which the ventricular cuff is attached. The seal assembly can be configured to extend into the ventricle. The seal assembly can include a base assembly, a ventricle opening member, and an adjustment assembly. The base assembly can be mounted to the retention assembly and configured for blocking blood flow through the blood flow channel. The ventricle opening member can be configured for filling an opening in a ventricular wall to which the ventricular cuff is attached. The ventricle opening member can be coupled to the base assembly via the adjustment assembly. The adjustment assembly can be operable to adjust a distance between the ventricle opening member and the base assembly.

In another aspect, an explant plug device is mountable to a ventricular cuff in fluid communication with a ventricle to block blood from flowing out of the ventricle through a blood flow channel of the ventricular cuff. The explant plug device includes a cap member and a retention assembly. The cap member includes a central portion and a perimeter portion that surrounds the central portion. The perimeter portion is configured for engaging a radially extending circumferential flange of the ventricular cuff to retain the cap member to the ventricular cuff. The cap member is configured to block blood flow through the blood flow channel. The retention assembly is configured for engagement with the perimeter portion to retain the perimeter portion in engagement with the radially extending circumferential flange of the ventricular cuff.

In some embodiments, the perimeter portion of the cap member includes a distally extending flange that extends distally from a perimeter edge portion of the central portion. The distally extending flange can include a return lip that extends towards the blood flow channel from a distal perimeter end of the distally extending flange. The distally extending flange and the return lip can be configured to engage the radially extending circumferential flange of the ventricular cuff to retain the cap member to the ventricular cuff. The distally extending flange can include a retention groove. The retention assembly can be configured for engagement with the retention groove to retain the retention assembly.

The retention assembly can have any suitable configuration. For example, the retention assembly can extend circumferentially around the perimeter portion. The retention assembly can include a first annular member, a second annular member, and a hinge that pivotally connects a first end of the first annular member with a first end of the second annular member. The retention assembly can be reconfigurable from a decoupled configuration to a coupled configuration in which the first annular member and the second annular member are coupled to surround the perimeter portion.

In some embodiments, the explant plug device further includes a blood flow channel assembly attached to the cap member. The blood flow channel assembly can be configured to extend through the blood flow channel of the ventricular cuff. The blood flow channel assembly can be detachably mounted to the cap member. The blood flow channel assembly can include an O-ring seal configured to interface with an inner surface of the ventricular cuff that defines the blood flow channel. The blood flow channel assembly can be configured for filling an opening in a ventricular wall to which the ventricular cuff is attached. The blood flow channel assembly can be configured to extend into the ventricle. The blood flow channel assembly can include a base assembly, a ventricle opening member, and an adjustment assembly. The base assembly can be mounted to the cap member. The ventricle opening member can be configured for filling an opening in a ventricular wall to which the ventricular cuff is attached. The ventricle opening member can be coupled to the base assembly via the adjustment assembly. The adjustment assembly can be operable to adjust a distance between the ventricle opening member and the base assembly.

In another aspect, an explant plug device is mountable to a ventricular cuff in fluid communication with a ventricle to block blood from flowing out of the ventricle through a blood flow channel of the ventricular cuff. The explant plug device includes a cap member. The cap member includes a central portion and three distally extending segments that extends distally from a respective perimeter edge portion of the central portion. The three distally extending segments are configured for engaging a radially extending circumferential flange of the ventricular cuff to retain the cap member to the ventricular cuff. Each of the distally extending segments includes a return lip that extends towards the blood flow channel from a distal end of the distally extending segment. Each of the distally extending segments and the respective return lip are configured to engage the radially extending circumferential flange of the ventricular cuff to retain the cap member to the ventricular cuff. The cap member is configured to block blood flow through the blood flow channel.

In some embodiments, the explant plug device further includes a blood flow channel assembly attached to the cap member. The blood flow channel assembly is configured to extend through the blood flow channel of the ventricular cuff. The blood flow channel assembly can be detachably mounted to the cap member. The blood flow channel assembly can include an O-ring seal configured to interface with an inner surface of the ventricular cuff that defines the blood flow channel. The blood flow channel assembly can be configured for filling an opening in a ventricular wall to which the ventricular cuff is attached. The blood flow channel assembly can be configured to extend into the ventricle. The blood flow channel assembly can include a base assembly, a ventricle opening member, and an adjustment assembly. The base assembly can be mounted to the cap member. The ventricle opening member can be configured for filling an opening in a ventricular wall to which the ventricular cuff is attached. The ventricle opening member can be coupled to the base assembly via the adjustment assembly. The adjustment assembly can be operable to adjust a distance between the ventricle opening member and the base assembly.

In another aspect, an explant plug device includes a cap member and a latching member. The cap member is configured to be mounted to a ventricular cuff in fluid communication with a ventricle to block blood from flowing out of the ventricle through a blood flow channel of the ventricular cuff. The latching mechanism includes a clip that is repositionable relative to the cap member from an unlocked position to a locked position. The clip includes two arms that are engaged with the ventricular cuff when the clip is in the locked position to retain the cap member to the ventricular cuff.

In some embodiments, the ventricular cuff includes an outward facing circumferential groove and ridges disposed at least partially within the outward facing circumferential groove. The two arms can be engaged with the at least one of the ridges when the clip is in the locked position. The blood flow channel can have a longitudinal axis. The clip can be configured to be moved transverse to the longitudinal axis, relative to the cap member, to move the clip from the unlocked position to the locked position. The clip can be configured to be moved perpendicular to the longitudinal axis, relative to the cap member, to move the clip from the unlocked position to the locked position.

In some embodiments, the latching mechanism defines channels along which the two arms travel during movement of the clip from the unlocked position to the locked position. The channels can define detents. When the cap member is not mounted to the ventricular cuff, movement of the clip from the unlocked position toward the locked position can engage the two arms into the detents to impede the clip from reaching the locked position. Each of the two arms can be configured to engage one of the detents independent of whether the other of the two arms engages one of the detents. In some embodiments, engagement of either of the two arms with one of the detents impedes the clip from reaching the locked position. In some embodiments, when the clip moves toward the locked position from the unlocked position and the cap member is mounted to the ventricular cuff, the ventricular cuff blocks engagement of the two arms with the detents.

In some embodiments, the two arms include teeth that are engaged with the at least one of the ridges when the clip is in the locked position. The teeth can be configured to engage the ventricular cuff when the clip is in the locked position to force the cap member into a fully seated position against the ventricular cuff. The ventricular cuff can include a circumferential flange. The ridges can be disposed on the circumferential flange. Each of the teeth can have a chamfered edge that engages the circumferential flange to force the cap member into the fully seated position against the ventricular cuff.

The clip can have any suitable configuration. For example, the clip can include a visual indicator. The visual indicator can be exposed when the clip is not in the locked position. The visual indicator can be configured to be obscured when the clip is in the locked position. The clip can include a latch that impedes movement of the clip from the locked position.

In some embodiments, the explant plug device further includes a sealing ring. The sealing ring can be configured to engage with an inward facing surface of the blood flow channel when the cap member is mounted to the ventricular cuff.

In another aspect, an explant plug device includes a blood flow blocking component and a retention clip. The explant plug device is mountable to a ventricular cuff in fluid communication with a ventricle to block blood from flowing out of the ventricle through the ventricular cuff. The ventricular cuff includes an outwardly facing annular flange, an outwardly facing annular groove, and a blood flow channel for an inlet cannula of a blood pump. The blood flow blocking component is configured to be mounted to the ventricular cuff to block flow of blood out of the ventricle via the ventricular cuff. The retention clip is coupled with the blood flow blocking component. The retention clip is repositionable relative to the blood flow blocking component from a retracted position that accommodates mounting of the blood flow blocking component to the ventricular cuff and a retention position that engages the outwardly facing annular flange to retain the blood flow blocking component to the ventricular cuff.

In many embodiments, the retention clip includes retention clip arms. The retention clip arms extend within retention clip arm passages of the blood flow blocking component. The retention clip arms are configured to extend into the outwardly facing annular groove when the retention clip is in the retention position.

In many embodiments, when the blood flow blocking component is not mounted onto the ventricular cuff, the retention clip is blocked from being repositioned to the retention position from the retracted position. The explant plug device can have any suitable configuration in which the retention clip is blocked from being repositioned to the retention position from the retracted position when the blood flow blocking component is not mounted onto the ventricular cuff.

In some embodiments, the explant plug device includes a lockout component pivotally coupled with the retention clip. The lockout component is held in a blocking orientation relative to the retention clip in which the lockout component blocks the retention clip from being repositioned to the retention position from the retracted position when the blood flow blocking component is not mounted onto the ventricular cuff. Mounting of the blood flow blocking component to the ventricular cuff reorients the lockout component relative to the retention clip from the blocking orientation to a non-blocking orientation that accommodates repositioning of the retention clip to the retention position from the retracted position. The retention clip can optionally include a spring arm that engages the lockout component to hold the lockout component in the blocking orientation when the blood flow blocking component is not mounted onto the ventricular cuff. The explant plug device can optionally include a recess configured to accommodate a protruding portion of the lockout component and engage the protruding portion of the lockout component to block the retention clip from being repositioned to the retention position from the retracted position when the lockout component is in the blocking orientation.

In some embodiments, the explant plug device includes a stop component. The stop component is biased to a blocking position when the blood flow blocking component is not mounted to the ventricular cuff. The stop component in the blocking position is configured to engage the retention clip to block repositioning of the retention clip to the retention position from the retracted position. Mounting of the blood flow blocking component to the ventricular cuff repositions the stop component from the blocking position to a non-blocking position that accommodates repositioning of the retention clip to the retention position from the retracted position. The explant plug device can optionally include a stop component spring that biases the stop component to the blocking position when the blood flow blocking component is not mounted to the ventricular cuff. The stop component can be configured to be engaged by the ventricular cuff during mounting of the blood flow blocking component to the ventricular cuff to displace the stop component from the blocking position to the non-blocking position.

In some embodiments, the explant plug device includes a two-part cam mechanism mounted to the retention clip. The two-part cam mechanism can be biased to a blocking configuration when the blood flow blocking component is not mounted to the ventricular cuff. The two-part cam mechanism in the blocking configuration can be configured to block repositioning of the retention clip to the retention position from the retracted position. Mounting of the blood flow blocking component to the ventricular cuff reconfigures the two-part cam mechanism from the blocking configuration to a non-blocking configuration that accommodates repositioning of the retention clip to the retention position from the retracted position. The explant plug device can optionally include a spring that engages the two-part cam mechanism to hold the two-part cam mechanism in the blocking configuration when the blood flow blocking component is not mounted onto the ventricular cuff.

The explant plug device can optionally include a spring arm member coupled with the blood flow blocking component. The spring arm member can include flexible arms configured to engage the retention clip arms in the retention position to inhibit repositioning of the retention clip relative to the blood flow blocking component. Each of the flexible arms can be configured to engage with a distal end portion of a respective one of the retention clip arms. Each of the distal end portions of the retention clip arms can optionally be shaped to inhibit repositioning of the retention clip from the retention position.

The retention clip can be constrained to be moved, relative to the ventricular cuff, from the retracted position to the retention position transverse to a longitudinal axis of the blood flow channel of the ventricular cuff. In some embodiments, the retention clip is constrained to be moved, relative to the ventricular cuff, from the retracted position to the retention position perpendicular to the longitudinal axis.

The retention clip can optionally include a visual indicator. In some embodiments, the visual indicator is disposed external to the blood flow blocking component when the retention clip is not in the retention position and is disposed within the blood flow blocking component when the retention clip is in the retention position.

For a fuller understanding of the nature and advantages of the present invention, reference should be made to the ensuing detailed description and accompanying drawings.

### BRIEF DESCRIPTION OF THE DRAWINGS

FIG. 1 is an exploded view illustration of a ventricular assist device and a ventricular cuff used to attach the ventricular assist device to a heart.

FIG. 2 is an exploded view illustration of the ventricular cuff of FIG. 1.

FIG. 3 is a cross-sectional view of the ventricular cuff of FIG. 1 attached to a ventricle wall.

FIG. 4 is a cross-sectional view of an explant plug device mounted to the ventricular cuff of FIG. 1 to block blood flow out of a ventricle through the ventricular cuff, in accordance with embodiments.

FIG. 5 and FIG. 6 are top-view schematic illustrations of a retention assembly of the explant plug device of FIG. 4.

FIG. 7 is a cross-sectional view of an embodiment of the explant plug device of FIG. 4 that includes a shorter blood flow channel seal assembly, in accordance with embodiments.

FIG. 8 is a cross-sectional view of an embodiment of the explant plug device of FIG. 4 that includes a longer blood flow channel seal assembly, in accordance with embodiments.

FIG. 9 is a cross-sectional view of an embodiment of the explant plug device of FIG. 4 that includes an attachable/detachable blood flow channel seal assembly, in accordance with embodiments.

FIG. 10, FIG. 11, and FIG. 12 illustrate different length embodiments of the attachable/detachable blood flow channel seal assembly of FIG. 9, in accordance with embodiments.

FIG. 13 is a cross-sectional view of an embodiment of the explant plug device of FIG. 4 that includes an adjustable height blood flow channel seal assembly, in accordance with embodiments.

FIG. 14 is a cross-sectional view of an adjustment mechanism of the adjustable height blood flow channel seal assembly of FIG. 13.

FIG. 15 is a cross-sectional view of another explant plug device mounted to the ventricular cuff of FIG. 1 to block blood flow out of a ventricle through the ventricular cuff, in accordance with embodiments.

FIG. 16 and FIG. 17 are top-view schematic illustrations of a retention assembly of the explant plug device of FIG. 15.

FIG. 18 is a cross-sectional view of an embodiment of the explant plug device of FIG. 15 that includes a blood flow channel seal assembly.

FIG. 19 is a cross-sectional view of another explant plug device mounted to the ventricular cuff of FIG. 1 to block blood flow out of a ventricle through the ventricular cuff, in accordance with embodiments.

FIG. 20 is a top-view schematic illustration of the explant plug device of FIG. 19.

FIG. 21 is a cross-sectional view of an embodiment of the explant plug device of FIG. 19 that includes a blood flow channel seal assembly.

FIG. 22 is an exploded view illustration of another embodiment of an explant plug device and a ventricular cuff used to attach a ventricular assist device to a heart, in accordance with embodiments.

FIG. 23 shows another view of the explant plug device of FIG. 22.

FIG. 24 shows a ventricular cuff to which the explant plug device of FIG. 22 can be mounted.

FIG. 25 is a perspective view of an attachment member of the ventricular cuff of FIG. 24.

FIG. 26 is a side cutaway view of an extension of the attachment member of FIG. 25.

FIG. 27, FIG. 28, and FIG. 29 illustrate a retention clip of the explant plug device of FIG. 22.

FIG. 30, FIG. 31, and FIG. 32 illustrate details of the explant plug device of FIG. 22.

FIG. 33 is another exploded view illustration of the explant plug device and the ventricular cuff of FIG. 22.

FIG. 34 illustrates an initial mounted configuration of the explant plug device and the ventricular cuff of FIG. 22.

FIG. 35 illustrates a latched mounted configuration of the explant plug device and the ventricular cuff of FIG. 22.

FIG. 36 is a cross-sectional view of a mounted configuration of an embodiment of the explant plug device and the ventricular cuff of FIG. 22.

FIG. 37 is an exploded view of an embodiment of the explant plug device of FIG. 22.

FIG. 38A is an oblique view of a base member of the explant plug device of FIG. 37.

FIG. 38B is a top view of the base member of FIG. 38A.

FIG. 39A is an oblique view of a retention clip of the explant plug device of FIG. 37.

FIG. 39B is a bottom view of the retention clip of FIG. 39A.

FIG. 39C is a top view of the retention clip of FIG. 39A.

FIG. 40A is a top view of retention related components of the explant plug device of FIG. 37.

FIG. 40B is a view of a coupling path for the retention related components of FIG. 40A.

FIG. 40C is a view of a lockout path for the retention related components of FIG. 40A.

FIG. 40D is a pair of pivoting components of the retention related components of FIG. 40A.

FIG. 40E is a top view of the retention related components of FIG. 40A in a lockout configuration.

FIG. 40F shows a close-up view of a lockout related portion of FIG. 40E.

FIG. 40G illustrates compression forces reacted by each of the pivoting components of FIG. 40D.

FIG. 41A is a top view of the retention related components of FIG. 40A in a coupling configuration.

FIG. 41B shows a close-up view of the retention related components of FIG. 40A in the coupling configuration.

FIG. 42A and FIG. 42B show a close-up views of interfacing portion of the retention clip and a spring arm member of the retention related components of FIG. 40A.

FIG. 43A is an oblique view of a base member of another embodiment of the explant plug device of FIG. 22.

FIG. 43B is a top view of the base member of FIG. 43A.

FIG. 44A is an oblique view of a retention clip of the explant plug device of FIG. 43A.

FIG. 44B is a bottom view of the retention clip of FIG. 44A.

FIG. 44C is a top view of the retention clip of FIG. 44A.

FIG. 45A is an oblique view of a stop component of the explant plug device of FIG. 43A.

FIG. 45B is a side view of the stop component of FIG. 45A.

FIG. 46A is a top view of retention related components of the explant plug device of FIG. 43A.

FIG. 46B is a close-up view of lockout-related components of the retention related components of FIG. 46A.

FIG. 47A is a top view of the retention related components of FIG. 46A in a lockout configuration.

FIG. 47B is a close-up view of lockout-related components of FIG. 47A in the lockout configuration.

FIG. 47C is a cross-sectional oblique view of the lockout-related components in the lockout configuration of FIG. 47B.

FIG. 47D is an oblique view of the lockout-related components in the lockout configuration of FIG. 47B.

FIG. 48A is a top view of the retention related components of FIG. 46A in a coupling configuration.

FIG. 48B is a close-up view of lockout-related components of FIG. 48A in the coupling configuration.

FIG. 48C is a cross-sectional oblique view of the lockout-related components in the coupling configuration of FIG. 48B.

FIG. 48D is an oblique view of the lockout-related components in the coupling configuration of FIG. 48B.

FIG. 49A is an oblique view of a base member of another embodiment of the explant plug device of FIG. 22.

FIG. 49B is a top view of the base member of FIG. 49A.

FIG. 50A is an oblique view of a retention clip assembly of the explant plug device of FIG. 49A.

FIG. 50B is a bottom view of the retention clip assembly of FIG. 50A.

FIG. 50C is a top view of the retention clip assembly of FIG. 50A.

FIG. 51A is a top view of retention related components of the explant plug device of FIG. 49A.

FIG. 51B is a close-up view of lockout-related components of the retention related components of FIG. 51A.

FIG. 51C is a top view of the retention related components of FIG. 51A in a retracted configuration.

FIG. 52A is a top view of the retention related components of FIG. 51A in a coupling/retention configuration.

FIG. 52B is a close-up view of the lockout-related components of FIG. 51B in a retention configuration of the retention related components.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

In some instances, a patient with an implanted ventricular assist device (VAD) recovers sufficiently to enable discontinuation of supplemental circulatory support provided by the VAD. In many of such instances, the patient experiences good long-term results following the discontinuation of the supplemental circulatory support. While current rates of discontinuation of supplemental circulatory support are low, the percentage of patients who can recover sufficiently to enable discontinuation of supplemental circulatory support may be underestimated. In one existing option for discontinuation of supplemental circulatory support, the VAD is turned off, the driveline is cut, and the VAD is left attached to the heart. In another existing option for discontinuation of supplemental circulatory support, the VAD is removed (explanted) from the patient and a ventricular patch plasty is performed to seal the ventricle.

Embodiments disclosed herein are directed to explant plug devices configured to replace a VAD attached to a ventricle via a ventricular cuff to block blood flow out of the ventricle through the ventricular cuff. In many embodiments, the explant plug device occupies less space than the VAD and therefore may provide associated benefits following explantation of the VAD. The explant plug device can be used to avoid having to perform a ventricular patch plasty during explantation of a VAD.

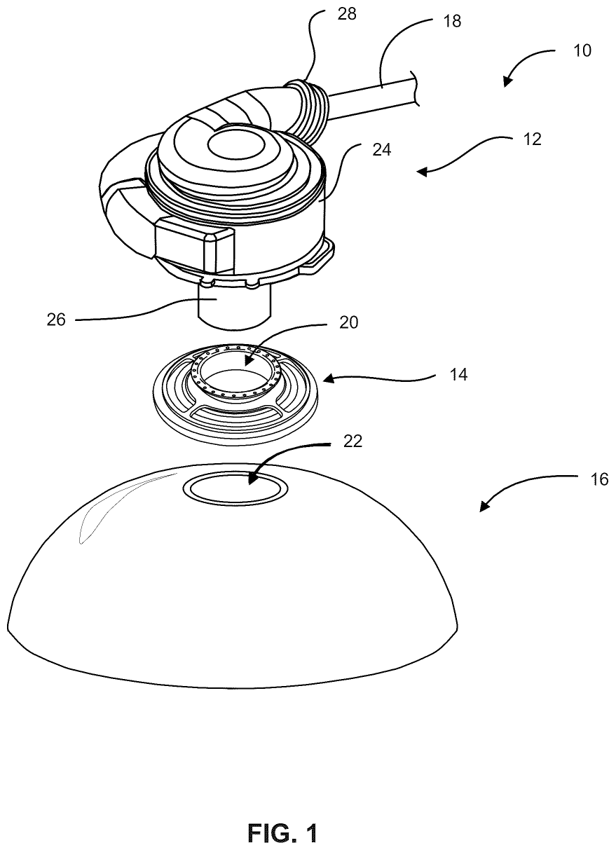

Turning now to the drawing figures in which similar reference identifiers are used to designate similar elements, FIG. 1 is an exploded view illustration of components of a VAD system 10, in accordance with embodiments. The illustrated components of the VAD system 10 include a VAD 12, a ventricular cuff 14 for coupling the VAD 12 with the heart 16 of a patient, and a percutaneous drive cable 18. The VAD 12 can be a left ventricular assist device (LVAD) for pumping blood from the left ventricle of the heart 16 to the aorta of the patient. The VAD 12 can be a right ventricular assist device (RVAD) for pumping blood from the right ventricle of the heart 16 to the pulmonary artery of the patient. The ventricular cuff 14 has a blood flow channel 20 and the heart 16 has a cored opening 22 via which the VAD 12 extracts blood from the ventricle for pumping to the aorta when the VAD 12 is an LVAD or to the pulmonary artery when the VAD 12 is an RVAD.

The VAD 12 can have any suitable configuration. In the illustrated embodiment, the VAD 12 includes a centrifugal blood pump. In some embodiments, the VAD 12 is configured the same or similar to the VAD embodiments described in PCT Patent Application PCT/US2015/019308, the contents of which are incorporated herein by reference. The VAD 12 includes a housing 24 (e.g., for housing an impeller), an inflow cannula 26, and an outlet 28. The inflow cannula 26 extends distally from the housing 24 for drawing blood from the ventricle of the heart 16 into the VAD 12. The VAD 12 pumps blood out of the outlet 28 for delivery to the aorta of the patient when the VAD 12 is an LVAD or to the pulmonary artery when the VAD 12 is an RVAD. The percutaneous driveline cable 18 is connected to the VAD 12 and configured to transfer drive signals and/or power to the VAD 12.

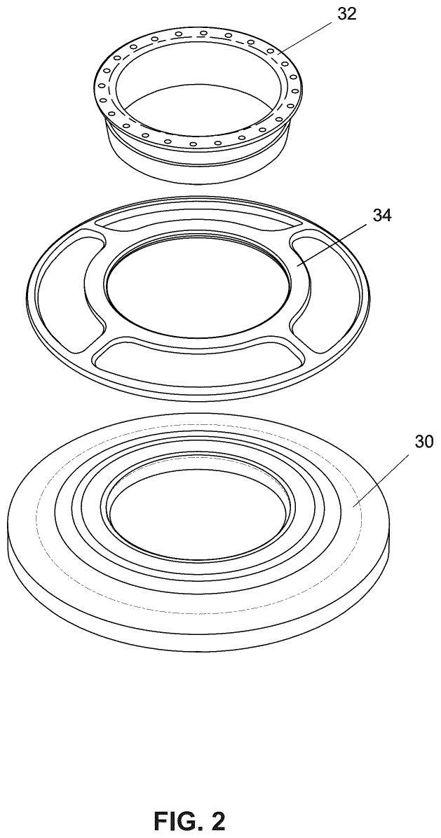

FIG. 2 is an exploded view illustration of the ventricular cuff 14. In the illustrated embodiment, the ventricular cuff 14 includes a suture ring 30, a blood flow channel member 32, and a support frame 34. The suture ring 30 can be formed from one or more suitable flexible materials (e.g., PTFE, polyester, silicone, rubber, or other textile or any combination thereof) and is configured to be sutured to the heart 16. The blood flow channel member 32 defines the blood flow channel 20. The blood flow channel member 32 can be formed from a suitable rigid or semi-rigid material so as to maintain the cored opening 22 in the heart 16. The support frame 34 is configured for coupling of the suture ring 30 and the blood flow channel 32 and restrains the shape of the suture ring 30 relative to the blood flow channel member 32. The ventricular cuff 14 can, however, have any alternate configuration that is suitable for attachment of the ventricular cuff 14 to the heart and has a blood flow channel via which the inflow cannula 26 of the VAD 12 is placed in fluid communication with the ventricle of the heart 16.

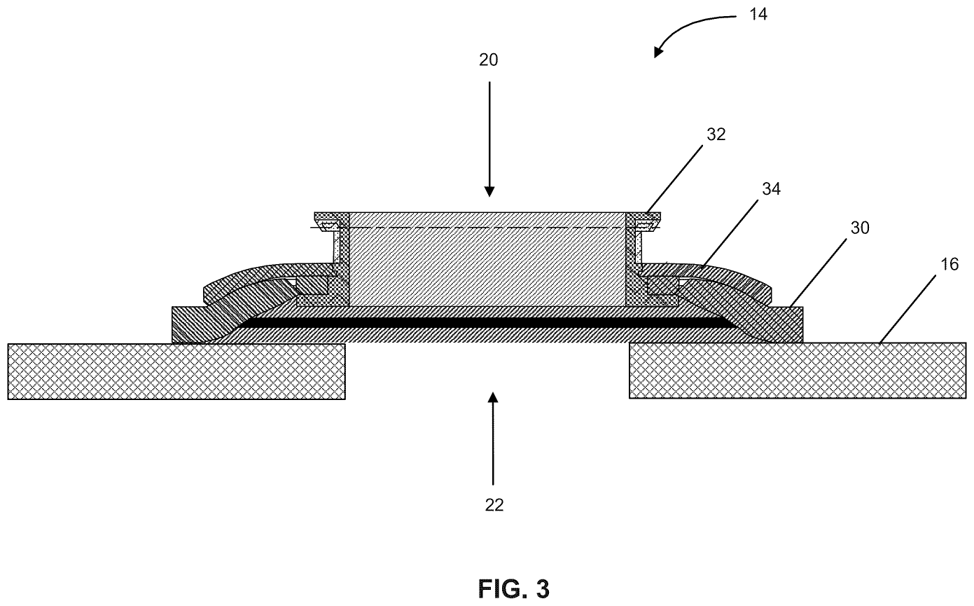

FIG. 3 is a cross-sectional view of the ventricular cuff 14 of FIG. 1 attached to a ventricle wall of the heart 16. As shown, without the VAD 12 coupled with the heart 16 via the ventricular cuff 14, the blood flow channel 20 of the ventricular cuff 14 and the cored opening 22 in the heart 16 form a blood flow path that must be blocked to prevent blood from escaping from the ventricle of the heart 16 out through the blood flow channel 20.

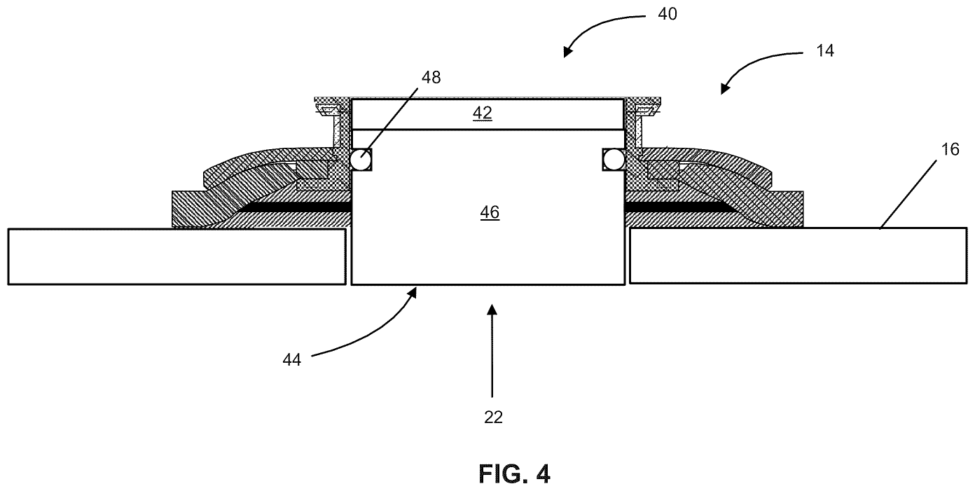

FIG. 4 is a cross-sectional view of an explant plug device 40 mounted to the ventricular cuff 14. The explant plug device 40 is configured to block blood flow out of a ventricle of the heart 16 through the ventricular cuff 14. The explant plug device 40 includes a retention assembly 42 and a blood flow channel seal assembly 44 coupled with the retention assembly 42. The blood flow channel seal assembly 44 is configured for insertion into the blood flow channel 20 of the ventricular cuff 14 and to block blood flow through the blood flow channel 20 from the ventricle of the heart 16. In the illustrated embodiment, the seal assembly 44 includes a seal assembly body member 46 and an O-ring seal 48 accommodated partially within an O-ring groove 50 in the body member 46. The body member 46 is attached to the retention assembly 42. The O-ring seal 48 protrudes a suitable amount from the O-ring groove 50 to engage the inner surface of the blood flow channel member 32 to block flow of blood out of the ventricle of the heart 16 through the ventricular cuff 14. The retention assembly 42 is reconfigurable between an insertion configuration that accommodates insertion of the explant plug device 40 into the blood flow channel 20 of the ventricular cuff 14 and a retention configuration in which the retention assembly 42 engages the inner surface of the blood flow channel member 32 to retain the explant plug device 40 to the ventricular cuff 14.

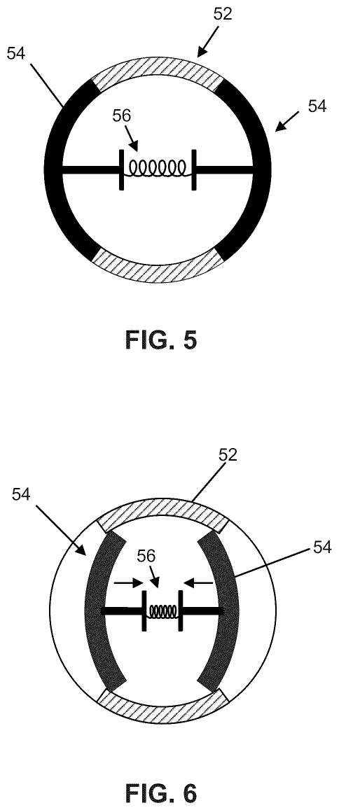

FIG. 5 and FIG. 6 are top-view schematic illustrations of an example embodiment of the retention assembly 42. FIG. 5 shows the retention assembly 42 in the retention configuration. FIG. 6 shows the retention assembly 42 in the insertion configuration. The retention assembly 42 can include a frame member 52, one or more retention members 54, and a spring assembly 56 coupled between the one or more retention members 54 and the frame member 52. The frame member 52 is connected to the seal assembly body member 46. The spring assembly 56 biases the position and/or orientation of the one or more retention members 54 to engage against the inner surface of the blood flow channel member 32 and thereby generate static frictional forces via which the explant plug device 40 is retained to the ventricular cuff 14. In the illustrated embodiment, there are two retention members 54 and the spring assembly 56 exerts a biasing force that pushes each of the two retention members 54 radially outward into engagement with the inner surface of the blood flow channel member 32. In an alternate embodiment, the retention assembly has a single retention member 54 and the spring assembly 56 exerts a biasing force and/or moment onto the single retention member 54 to engage the retention member 54 with the inner surface of the blood flow channel member 32. To reconfigure the retention assembly 42 from the retention configuration to the insertion configuration, the one or more retention members 54 are moved from the position(s) shown in FIG. 5 to the position(s) shown in FIG. 6, which is accommodated via a corresponding reconfiguration of the spring assembly 56. In the illustrated embodiment, the spring assembly 56 comprises a compression spring that is preloaded in compression in the retention configuration and has an increased compressive preload in the insertion configuration relative to the retention configuration. The spring assembly 56 can include any suitable number of springs and/or type of springs. The spring assembly 56 can include one or more preloaded springs that are configured to hold each of the one or more retention members in engagement with the inner surface of the ventricular cuff 14 in the retention configuration. In some embodiments, the one or more retention members 54 include a first retention member 54 and a second retention member 54. The first retention member 54 can be configured for engaging a first annular portion of the inner surface of the blood flow channel member 32 in the retention configuration. The second retention member 54 can be configured for engaging a second annular portion of the inner surface of the blood flow channel member 32 in the retention configuration. The second annular portion of the inner surface of the blood flow channel member 32 can be disposed opposite to the first annular portion of the inner surface of the blood flow channel member 32. In many embodiments, the retention assembly 42 includes the frame member 52 and the seal assembly 44 is attached to the frame member 52. Each of the one or more retention members 54 can be coupled with the frame member 52 to accommodate movement of the retention member 54 relative to the frame member 52.

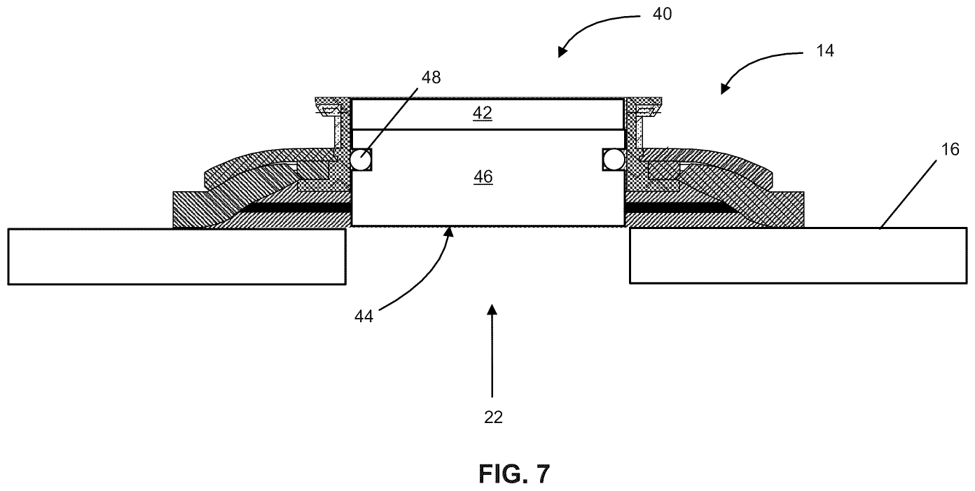

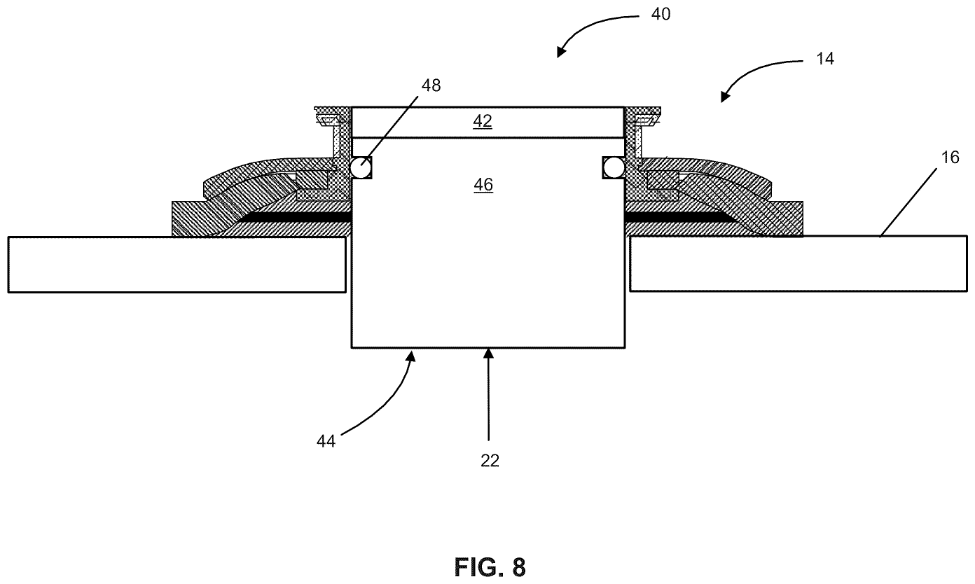

The height of the seal assembly 44 can be selected to provide a desired amount of extension of the seal assembly 44 towards the ventricle. For example, in the embodiment shown in FIG. 4 the seal assembly 44 assembly extends to fill the cored opening 22 in the heart 16. Employing a seal assembly 44 that extends to fill the cored opening 22 may serve to keep the cored opening 22 open to accommodate potential reimplantation of a VAD 12. FIG. 7 shows an embodiment of the explant plug device 40 that includes a shorter blood flow channel seal assembly 44 that does not extend to fill the cored opening 22 in the heart 16. Employing a seal assembly 44 that does not extend to fill the cored opening 22 may serve to accommodate tissue ingrowth into the cored opening 22 over time to help increase the capability of the heart wall in the vicinity of the cored opening 22 to contribute to pumping blood from the ventricle. FIG. 8 shows an embodiment of the explant plug device 40 that includes a longer blood channel seal assembly 44 that extends into the ventricle of the heart 16. The length of the seal assembly 44 can also be selected to accommodate the specific applicable dimensions of the heart 16, which varies for different patients.

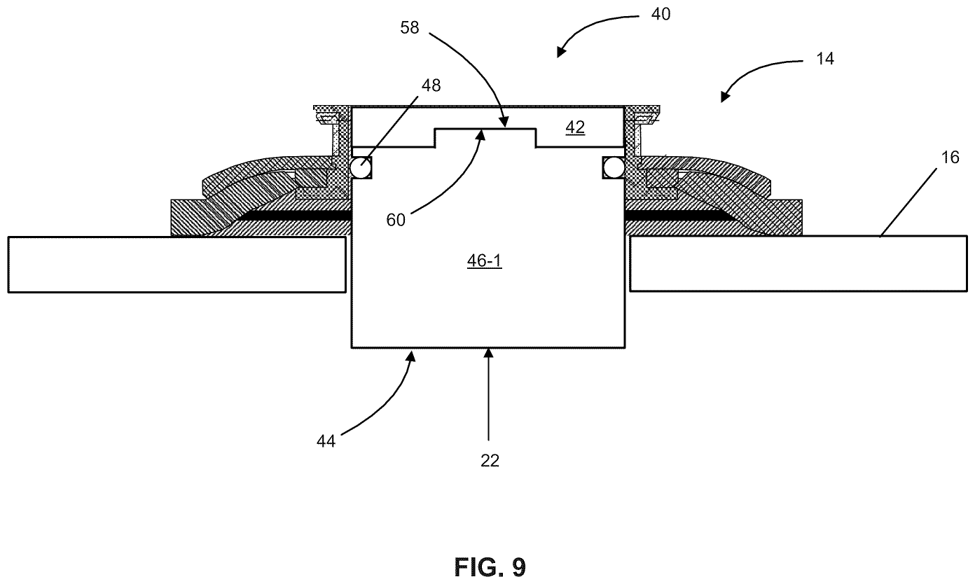

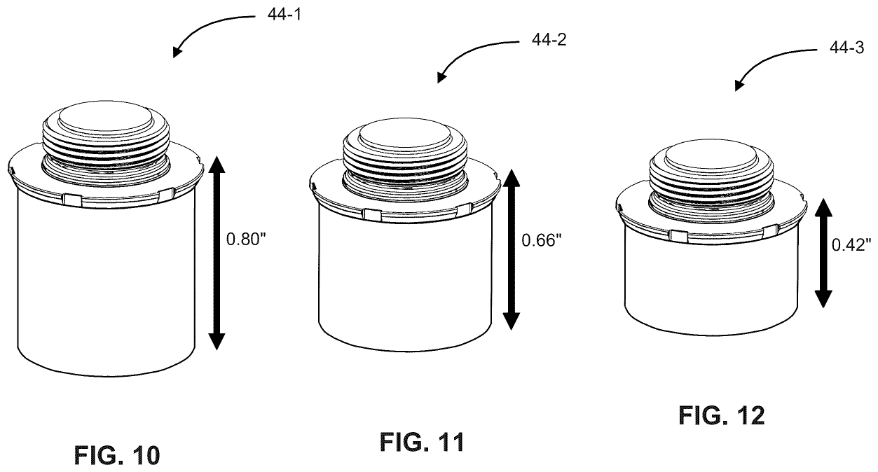

In some embodiments of the explant plug device 40, the seal assembly 44 is detachably mountable to the retention assembly 42. For example, in the embodiment of the explant plug device 40 shown in FIG. 9, the seal assembly 44 has a protruding male threaded attachment feature 58 and the frame member 52 of the retention assembly 42 has a female threaded recess 60 into which the protruding male threaded attachment feature 58 can be threaded to mount the seal assembly 44 to the retention assembly 42. FIG. 10, FIG. 11, and FIG. 12 illustrate example embodiments 44-1, 44-2, 44-3 of the attachable/detachable blood channel seal assembly of FIG. 9 with different heights.

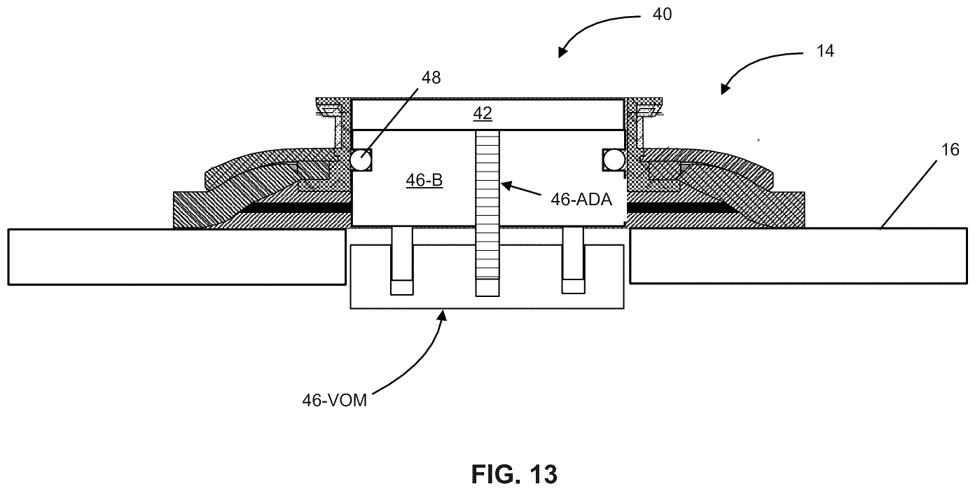

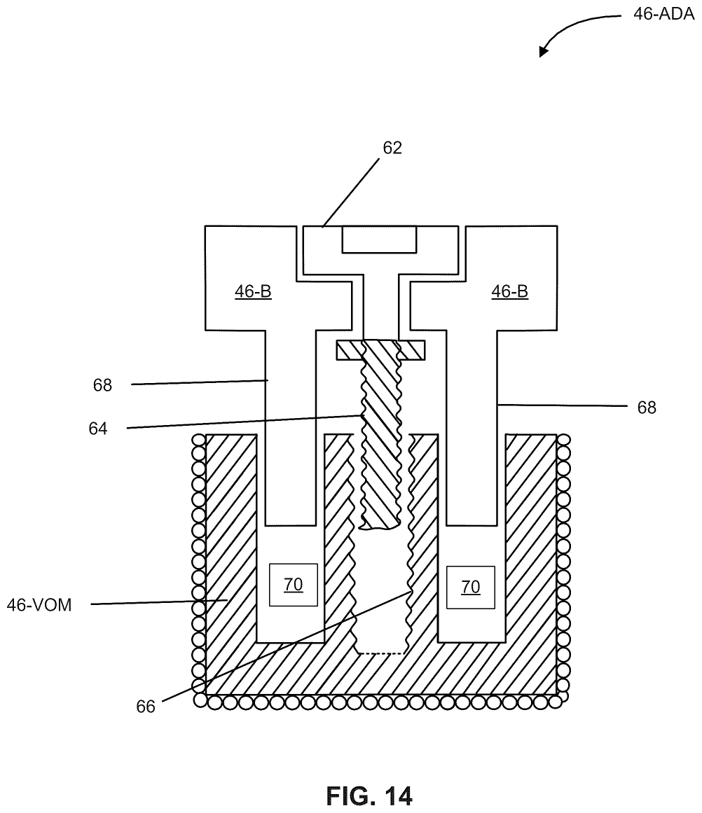

FIG. 13 shows an embodiment of the explant plug device 40 that includes an adjustable height blood flow channel seal assembly 44-ADJ. The adjustable height blood flow channel seal assembly 44-ADJ includes a base assembly 46-B, a ventricle opening member 46-VOM, and an adjustment assembly 46-ADA. The base assembly 46-B is mounted to the retention assembly 42. The base assembly 46-B is configured for blocking blood flow through the blood flow channel 20 of the ventricular cuff 14. The ventricle opening member 46-VOM is configured for filling the cored opening 22 of the heart 16. The ventricle opening member 46-VOM is coupled to the base assembly 46-B via the adjustment assembly 46-ADA. The adjustment assembly 46-ADA is operable to adjust a distance between the ventricle opening member 46-VOM and the base assembly 46-B. FIG. 14 is a cross-sectional view of an embodiment of the adjustment assembly 46-ADA that includes a captive adjustment member 62 with male threads 64. The ventricle opening member 46-VOM includes female threads 66 engaged by the male threads 64 of the captive adjustment member 62. The base assembly 46-B includes guide rods 68 and the ventricle opening member 46-VOM includes guide apertures 70 into which the guide rods 68 extend to maintain rotational alignment between the base assembly 46-B and the ventricle opening member 46-VOM while accommodating changing of a separation distance between the base assembly 46-B and the ventricle opening member 46-VOM. Selective rotation of the captive adjust member 62 relative to the base assembly 46-B is used to selectively adjust the separation distance between the base assembly 46-B and the ventricle opening member 46-VOM.

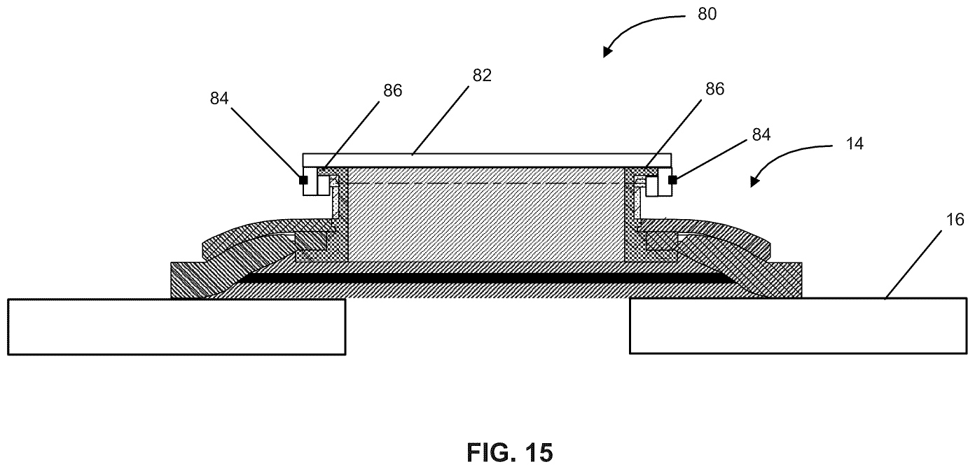

FIG. 15 is a cross-sectional view of another explant plug device 80 mounted to the ventricular cuff 14 to block blood flow out of a ventricle through the ventricular cuff 14, in accordance with embodiments. The explant plug device 80 includes a cap member 82 and a retention assembly 84. The cap member 82 includes a central portion and a perimeter portion that surrounds the central portion. The perimeter portion is configured for engaging a radially extending circumferential flange 86 of the ventricular cuff 14 to retain the cap member 82 to the ventricular cuff 14. In some embodiments, the cap member 82 is configured to block blood flow through the blood flow channel 20. The retention assembly 84 is configured for engagement with the perimeter portion to retain the perimeter portion in engagement with the radially extending circumferential flange 86 of the ventricular cuff 14. The perimeter portion of the cap member 82 includes a distally extending flange that extends distally from a perimeter edge portion of the central portion of the cap member 82. The distally extending flange 86 includes a return lip that extends towards the blood flow channel 20 from a distal perimeter end of the distally extending flange. The distally extending flange and the return lip are configured to engage the radially extending circumferential flange 86 of the ventricular cuff 14 to retain the cap member 82 to the ventricular cuff 14. The distally extending flange 86 can include comprises a retention groove configured to be engaged by the retention assembly 84 to retain the retention assembly 84 to the cap member 82. The retention assembly 84 can extend circumferentially around the perimeter portion of the cap member 82.

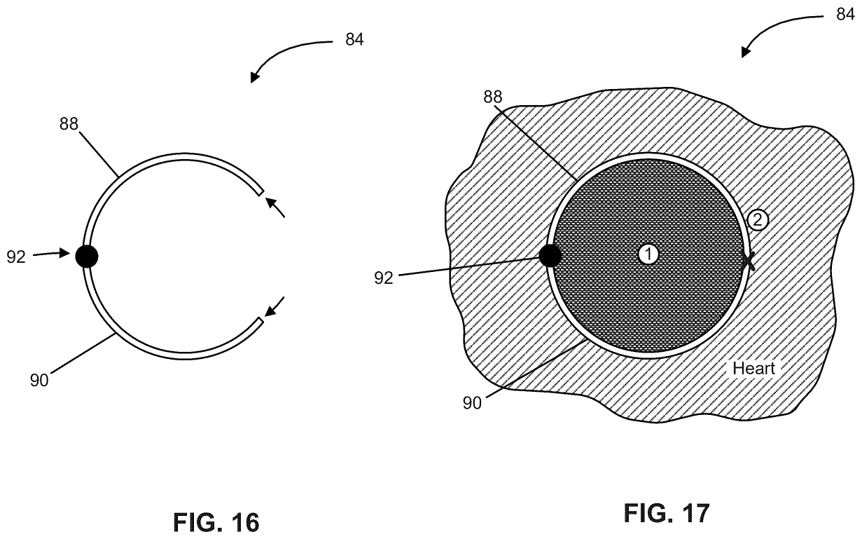

FIG. 16 and FIG. 17 are top-view schematic illustrations of an embodiment of the retention assembly 84 of the explant plug device 80. The embodiment of the retention assembly 84 includes a first annular member 88, a second annular member 90, and a hinge 92 that pivotally connects a first end of the first annular member 88 with a first end of the second annular member 90. The embodiment of the retention assembly 84 is reconfigurable from a decoupled configuration (shown in FIG. 16) to a coupled configuration (shown in FIG. 17) in which the first annular member 88 and the second annular member 90 are coupled to surround the perimeter portion of the cap member 82 to retain the cap member 82 to the ventricular cuff 14.

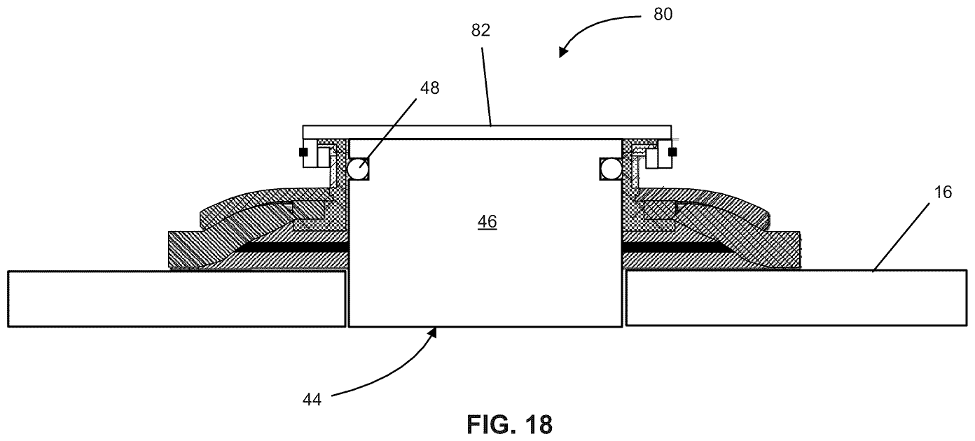

FIG. 18 is a cross-sectional view of an embodiment of the explant plug device 80 that includes the blood flow channel seal assembly 44 described herein with respect to the explant plug device 40. For example, the blood flow channel seal assembly 44 can be configured to extend at least partially through the blood flow channel 20 of the ventricular cuff 14. The blood flow channel seal assembly 44 can be configured to be detachably mounted to the cap member 82. The blood flow channel seal assembly 44 can include the O-ring seal 48 configured to interface with an inner surface of the ventricular cuff 14 that defines the blood flow channel 20. The blood flow channel seal assembly 44 can be configured for filling the cored opening 22 in the heart 16. The blood flow channel assembly 44 can be configured to extend into the ventricle. The blood flow channel assembly 44 can be the adjustable blood flow channel assembly 44-ADJ shown in FIG. 13.

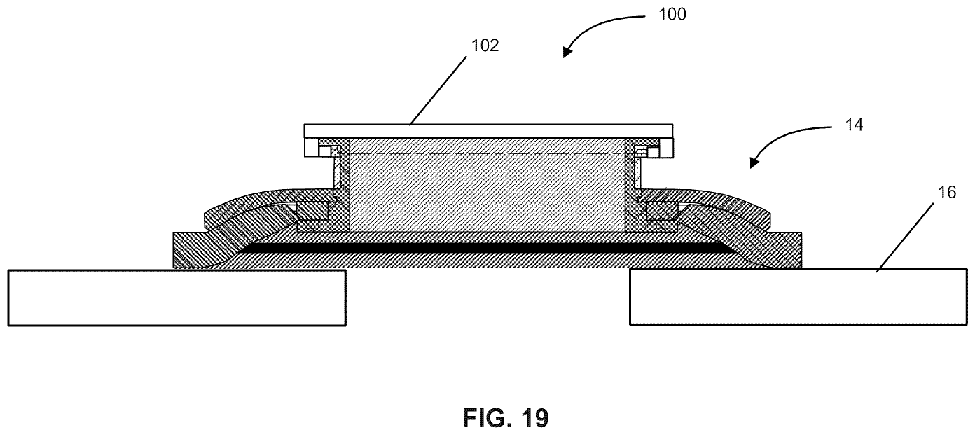

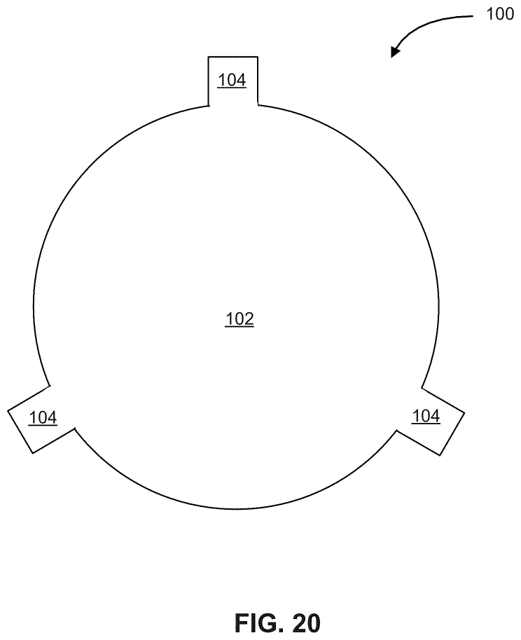

FIG. 19 is a cross-sectional view of another explant plug device 100 mounted to the ventricular cuff 14 to block blood flow out of a ventricle through the ventricular cuff 14, in accordance with embodiments. FIG. 20 shows a top view of the explant plug device 100. The explant plug device 100 includes a cap member 102. The cap member 102 includes a central portion and three distally extending segments 104. Each of the distally extending segments 104 extends distally from a respective perimeter edge portion of the central portion of the cap member 102. The distally extending segments 104 are configured for engaging the radially extending circumferential flange 86 of the ventricular cuff 14 to retain the cap member 102 to the ventricular cuff 14. Each of the distally extending segments 104 includes a return lip that extends towards the blood flow channel 20 of the ventricular cuff 14. The cap member 102 is configured to block blood flow through the blood flow channel 20.

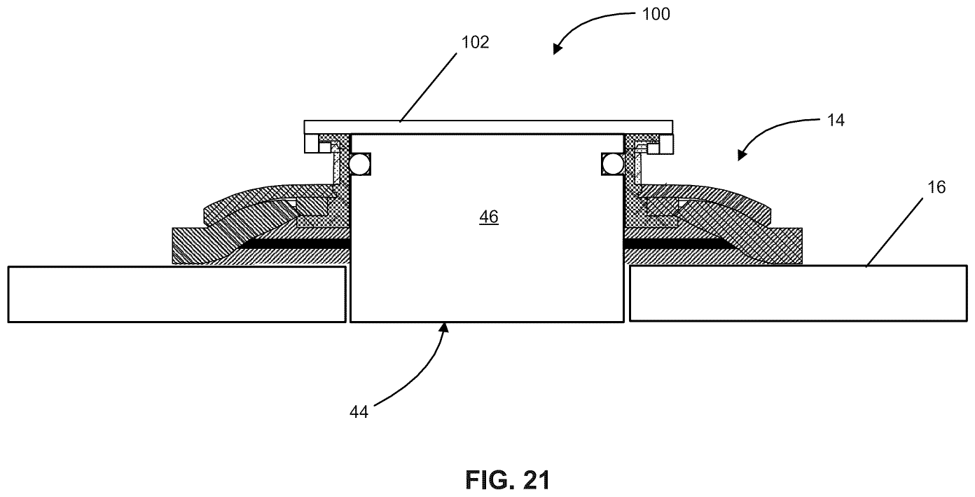

FIG. 21 is a cross-sectional view of an embodiment of the explant plug device 100 that includes the blood flow channel seal assembly 44 described herein with respect to the explant plug device 44. For example, the blood flow channel seal assembly 44 can be configured to extend at least partially through the blood flow channel 20 of the ventricular cuff 14. The blood flow channel seal assembly 44 can be configured to be detachably mounted to the cap member 102. The blood flow channel seal assembly 44 can include the O-ring seal 48 configured to interface with an inner surface of the ventricular cuff 14 that defines the blood flow channel 20. The blood flow channel seal assembly 44 can be configured for filling the cored opening 22 in the heart 16. The blood flow channel seal assembly 44 can be configured to extend into the ventricle. The blood flow channel seal assembly 44 can be the adjustable blood flow channel assembly 44-ADJ shown in FIG. 13.

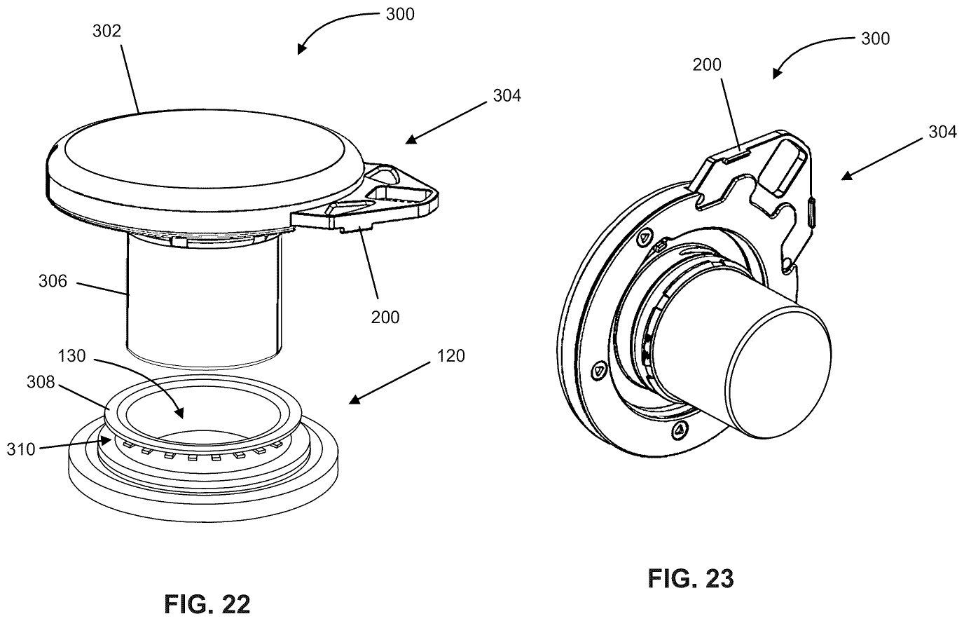

FIG. 22 shows explant plug devices 300, 600, 1200, 1900 and a ventricular cuff 120 to which the explant plug devices 300, 600, 1200, 1900 can be mounted to block blood flow out of the ventricle through the ventricular cuff 120. In many embodiments, the ventricular cuff 120 is configured as described for the ventricular cuff 120 disclosed in U.S. Pat. No. 9,144,637, the full disclosure of which is incorporated herein by reference. The ventricular cuff 120 includes an outwardly facing annular flange 308 and defines an outwardly facing annular groove 310. FIG. 23 shows another view of the explant plug device 300, 600, 1200, 1900. Each of the explant plug devices 300, 600, 1200, 1900 can include a cap assembly 302, a latching mechanism 304, and a blood flow channel seal assembly 306. The explant plug devices 300, 600, 1200 1900 are configured to be mounted to the ventricular cuff 120 to block blood from flowing out of the ventricle through a blood flow channel 130 of the ventricular cuff 120. The latching mechanism 304 includes a clip 200 that is repositionable relative to the cap assembly 302 from an unlocked position to a locked position. The clip 200 includes two arms 214 that are engaged with the ventricular cuff 120 when the clip 200 is in the locked position to retain the cap assembly 302 to the ventricular cuff 120. The blood flow channel seal assembly 306 is configured to be inserted into a blood flow channel 130 of the ventricular cuff 120.

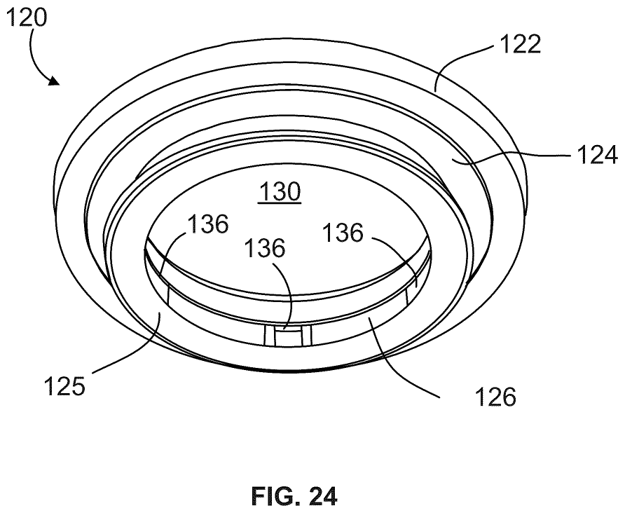

FIG. 24 shows the ventricular cuff 120. The ventricular cuff 120 includes an annular fastening member 122, a linking member 124, and an attachment member 126. The fastening member 122 can be sutured to heart tissue, and can include, for example, a fabric such as PTFE felt.

The linking member 124 can be formed of, for example, an elastomer such as silicone, and can include a reinforcement member such as a mesh ring. The linking member 124 is disposed about an outer circumference of the attachment member 126 and serves as a linking member to couple the attachment member 126 to the fastening member 122, as discussed further below. The linking member 124 can be coupled to the fastening member 122 by, for example, sutures. The linking member 124 can also be molded directly to the fastening member 122. The linking member 124 includes a bottom surface 125 configured to be engaged to form a face seal with the cap assembly 302.

The blood flow channel seal assembly 306 can include a first circumferential taper 156 that engages extensions 136 of the attachment member 126 and deflects the extensions 136 outwardly as the blood flow channel seal assembly 306 advances through the blood flow channel 130. The blood flow channel seal assembly 306 can include a second circumferential taper 158 and can define a circumferential groove 160 between the second circumferential taper 158 and the cap assembly 302.

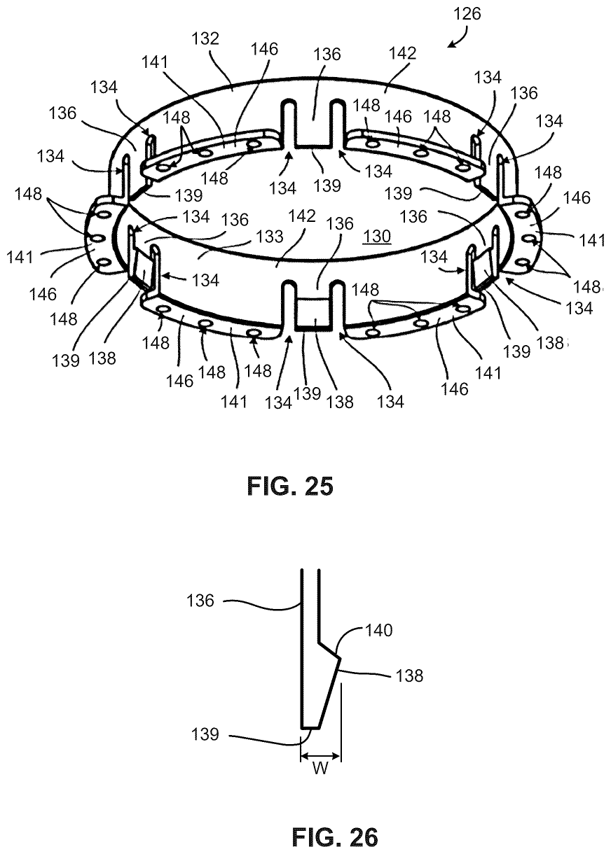

FIG. 25 is a perspective view of the attachment member 126. FIG. 26 is a side cutaway view of an extension of the attachment member 126. The attachment member 126 can be formed of, for example, a rigid material such as metal. The attachment member 126 includes a ring portion 132 having a wall 133 with cutouts 134 that define flexible extensions 136. Each extension 136 includes a lower tapered portion 138 (FIG. 26) disposed on a free end 139 of the extension 136, facing inward toward the blood flow channel 130. As the first circumferential taper 156 of the blood flow seal assembly 306 is inserted into the blood flow channel 130, the lower tapered portions 138 engage the first circumferential taper 156, causing the extensions 136 to flex outward from the blood flow channel 130 and permit the first circumferential taper 156 to pass through the blood flow channel 130. When the lower tapered portions 138 are disposed in the circumferential groove 160, the lower tapered portions 138 engage the second circumferential taper 158 of the blood flow channel seal assembly 306 to inhibit removal of the blood flow channel 306 from the blood flow channel 130. Each lower tapered portion 138 includes upper tapered portion 140, and the width of each lower tapered portion 138, W, decreases along the length of each lower tapered portion 138, between the upper tapered portion 140 and the free end 139.

The extensions 136 can have equal sizes or can be selected to have differing sizes. For example, asymmetrical lengths of the extensions 136 can cause the extensions 136 to engage the circumferential tapers 156, 158 sequentially rather than consecutively during travel of the blood flow channel seal assembly 306 relative to the ventricular cuff 120, reducing the force required to couple the explant plug device 300 to the ventricular cuff 120 or to uncouple the explant plug device 300 from the ventricular cuff 120.

The attachment member 126 includes flanged portions 146, disposed between the extensions 136 along the outer circumference of the attachment member 126, at the bottom 141 of the attachment member 126. The flanged portions 146 extend generally perpendicular to the wall 133. When the explant plug device 300 is coupled to the ventricular cuff 20, the flanged portions 146 are disposed in a plane generally parallel to a circumferential flange 162 of the cap assembly 302. When the explant plug device 300 is locked to the ventricular cuff 20, the flanged portions 146 are captured between the clip 200 and the circumferential flange 162, blocking demounting of the explant plug device 300 from the ventricular cuff 120.

To attach the explant plug device 300 to the ventricular cuff, the blood flow channel seal assembly 306 is inserted into the blood flow channel 130. As the blood flow channel seal assembly 306 is advanced through the blood flow channel 130, the first circumferential taper 156 passes the upper tapered portion 140 of the lower tapered portions 138. The engagement of the lower tapered portions 138 with the first circumferential taper 156 (which resists advancement of the cannula 150 by deflecting the extensions 136) ends abruptly, permitting the extensions 136 to straighten so that the lower tapered portions 138 reside in the circumferential groove 160. The sudden decrease in resistance to advancement of the blood flow channel seal assembly 306 produces a tactile snap-like sensation, indicating that the blood flow channel seal assembly 306 is coupled to the ventricular cuff 120. The upper tapered portion 140 of the lower tapered portions 138 engage the second circumferential taper 158, impeding detachment of the explant plug device 300 from the ventricular cuff 120. The bottom surface 125 of the linking member 124 engages the circumferential flange 162 of the cap assembly 302, limiting further advancement of the explant plug device 300 relative to the ventricular cuff 120.

After the explant plug device 300 and the ventricular cuff 120 are coupled, the explant plug device 300 can be separated from the ventricular cuff 120 by a force sufficient to deflect the extensions 136. Engagement of the upper tapered portions 140 with the second circumferential taper 158 deflects the extensions 136, allowing the explant plug device to be detached from the ventricular cuff 120.

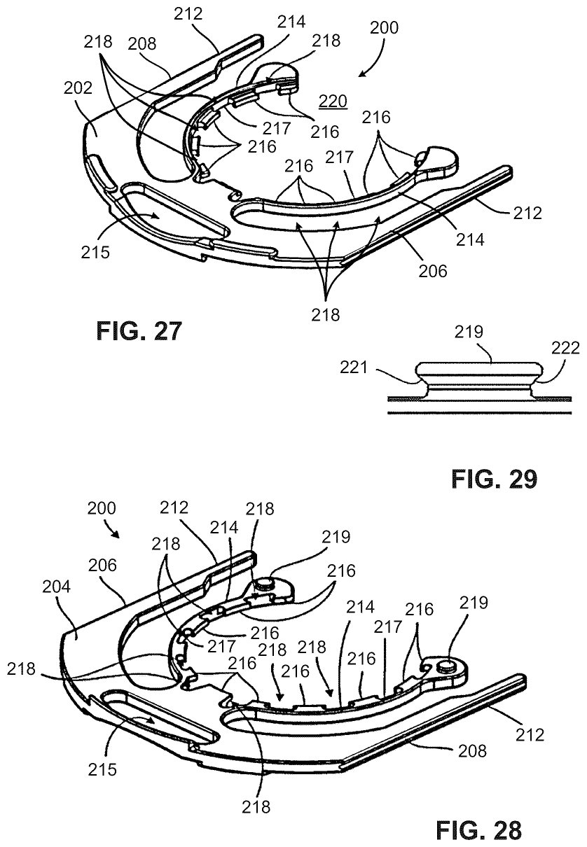

FIG. 27, FIG. 28, and FIG. 29 illustrate the clip 200 of the explant plug device 300. The clip 200 is used to retain the explant plug device 300 to the ventricular cuff 120. When the clip 200 is in the unlocked position, the clip 200 is positioned to accommodate insertion of the cap assembly 302 into the blood flow channel 130 of the ventricular cuff 120. When the clip 200 is in the locked position, the clip 200 is engaged with an outwardly extending flange of the ventricular cuff 120 and blocks demounting of the explant plug device 300 from the ventricular cuff 120. The clip 200 includes a top side 202, a bottom side 204, and opposite lateral sides 206, 208. The clip 200 can be formed of, for example, a rigid plastic, such as PEEK, or metal, such as titanium.

The clip 200 includes guide rails 212 and arms 214. The clip 200 defines a recess or opening 215. The guide rails 212 guide the clip 200 through a linear motion as the clip 200 is moved relative to the cap assembly 302. The opening 215 is configured to receive a tool or a finger of a clinician to facilitate disengagement of the clip 200 from the outwardly extending flange of the ventricular cuff 120 via movement of the clip 200 relative to the cap assembly 302 from its locked position. The arms 214 are curved and resilient. The arms 214 define an opening 220. As the clip 200 moves relative to the cap assembly 302, the cap assembly 302 forces the arms 214 laterally outward, expanding the opening 220 and allowing the arms 214 to extend around the ventricular cuff 120. In the locked position of the clip 200, the cap assembly 302 forces the arms 214 laterally inward to engage the outwardly extending flange of the ventricular cuff 120 so secure the explant plug device 300 to the ventricular cuff 120.

The arms 214 include teeth 216 that extend from inner walls 217 of the arms 214 toward the opening 220. In the locked position of the clip 200, the teeth 216 are disposed under the outwardly extending flange of the ventricular cuff 120 thereby blocking detachment of the explant plug device 300 from the ventricular cuff 120. Between the teeth 216 are gaps 218 that permit the arms 214 to flex laterally as the clip 200 is moved relative to the cap assembly 302. When the clip 200 is in a locked position about the ventricular cuff 120, the teeth 216 engage the ventricular cuff 120 to inhibit rotation of the explant plug device 300 relative to the ventricular cuff 120.

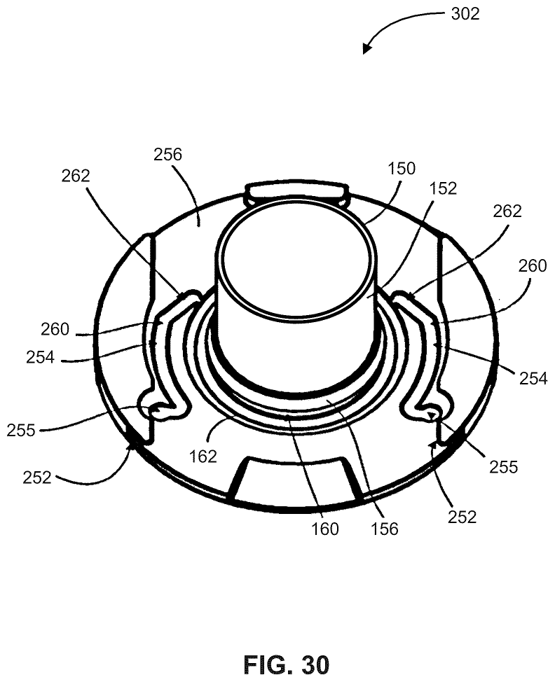

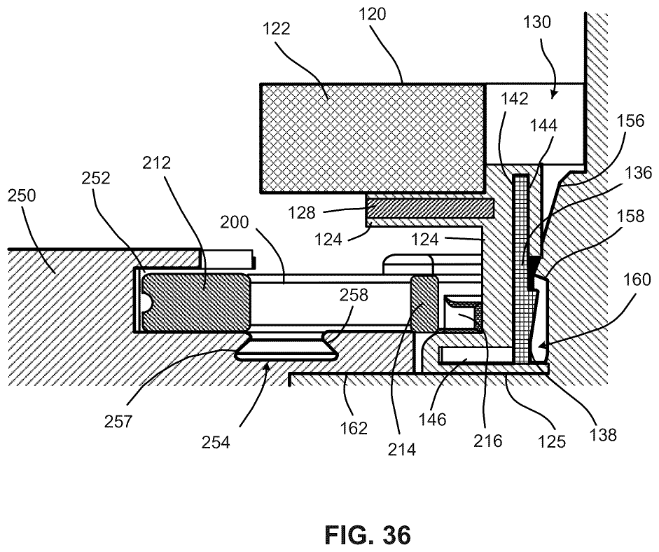

Each arm 214 includes a post 219 extending from the bottom side 204 that is received in one of the channels 254 (FIG. 30) defined by the cap assembly 302. As the cap assembly 302 receives the clip 200, the posts 219 travel through the channels 254, directing the lateral flexion of the arms 214. The posts 219 each include angled walls 221, 222 (FIG. 29) that engage angled walls 257, 258 (FIG. 36) of the cap assembly 302 that define the channels 254, capturing the posts 219 in the channels 254.

Referring to FIG. 30, the cap assembly 302 defines generally parallel slots 252 that receive the guide rails 212 of the clip 200. The cap assembly 302, in a top side 256, also defines the channels 254 that receive the posts 219 between the angled walls 257, 258 (FIG. 36). The angled walls 257, 258 capture the posts 219, impeding the posts 219 from leaving the channels 254 and maintaining the arms 214 in a plane above the top side 256.

The cap assembly 302 defines an entry recess 255 at each channel 254 that admits the post 219. The distance between the entry recesses 255 is larger than the distance between the posts 219 when the arms 214 of the clip 200 are not flexed.

To insert the posts 219 into the channels 254, the clinician flexes the arms 214 outward, loading the resilient arms 214 and permitting the posts 219 to enter the channels 254 at the entry recesses 255. After the posts 219 are positioned in the entry recesses 255, the arms 214 flex inward to their natural resting condition, moving the posts 219 in the channels 254 away from the entry recesses 255. Because the posts 219 are captured in the channels 254, the clip 200 will not separate from the cap assembly 302 until the clinician flexes the arms 214 outward and upward, permitting the posts 219 to leave the channels 254 at the entry recesses 255. The cap assembly 302 can be provided with the clip 200 already positioned in the channels 254, and thus already captured by the cap assembly 302, to streamline mounting of the explant plug device 300 to the ventricular cuff 120.

A first portion 260 of the channels 254 curves outward to spread the arms 214, permitting the arms 214 to extend around the ventricular cuff 14. A second portion 262 of the channels 254 curves inward toward the ventricular cuff 14, moving the arms 214 inward about the ventricular cuff 120.

Referring to FIG. 31, the guide rails 212 of the clip 200 enter the slots 252, and the posts 219 are captured in the channels 254. The clip 200 travels in a generally linear direction relative to the cap assembly, in the direction of arrow I₁, until the clip 200 reaches the position of FIG. 32. As the clip 200 is advanced into the cap assembly 302, the force in the direction of arrow I₁ causes the posts 219 to deflect outward in the channels 254. Once the posts 219 have reached the peak distance between the channels 254, the insertion force required in the direction of arrow I₁ lessens as the inward deflection force of the arms 214 drive the clip 200 through the second portion 262 of the channels 254. The clip 200 travels linearly as the posts 219 travel through the channels 254, until the position of FIG. 32 is reached in which the arms 214 are in their relaxed position.

To move the clip 200 back to the unlocked position, the clip 200 is retracted in a direction opposite the arrow I₁, and the posts 219 travel in the opposite direction through the channels 254. During removal of the clip 200, the second portion 262 expands the arms 214 and the first portion 260 permits the arms 214 to become closer together. The angle of the first portion 260 is less steep than the angle of the second portion 262, which results in the force to remove the clip 200 being higher than the force to move the clip 200 into the locking position.

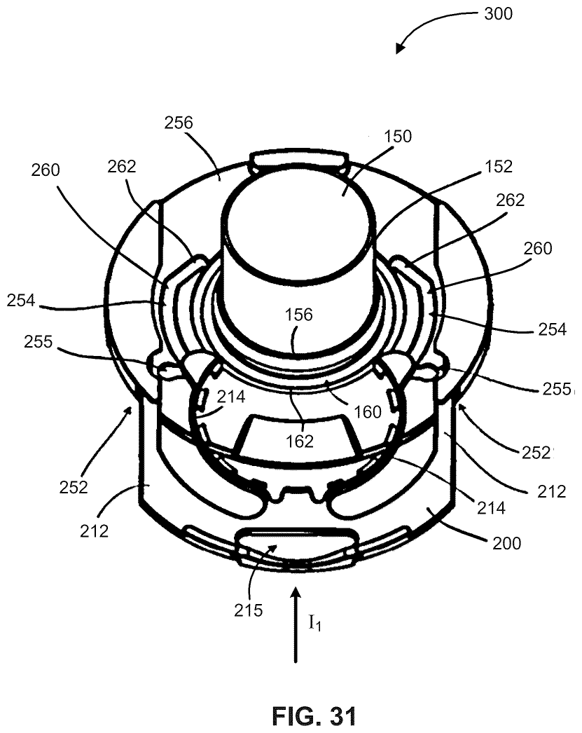

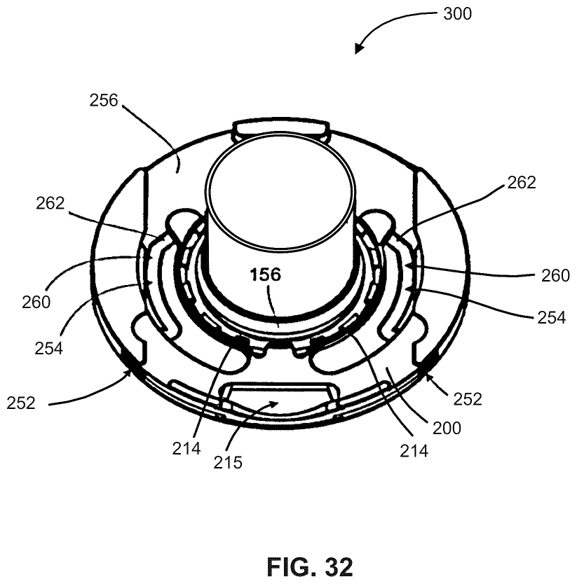

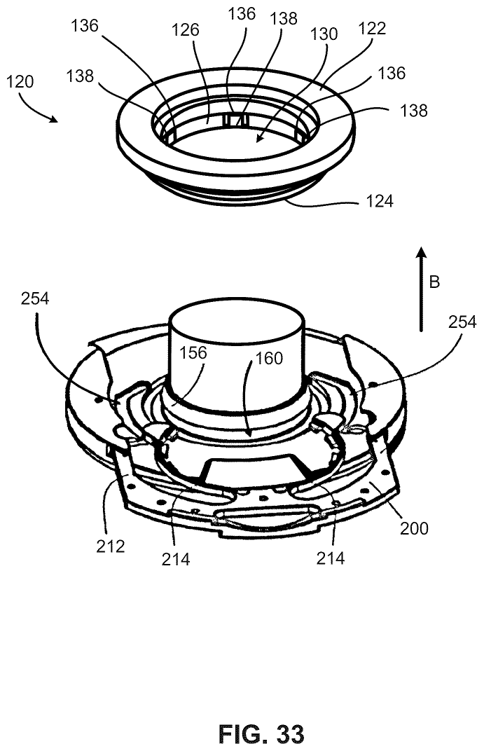

Referring to FIG. 33, a clinician moves the explant plug device 300 relative to the ventricular cuff 120, in the direction of arrow B, so that the proximal portion 152 enters the opening 130 of the ventricular cuff 120. As the explant plug device 300 is mounted to the ventricular cuff 120, the first circumferential taper 156 of the explant plug device 300 deflects the extensions 136 away from the explant plug device 300. The first circumferential taper 156 and the second circumferential taper 158 advance past the tapered portions 138 of the extensions 136. As the first circumferential taper 156 advances past the tapered portions 138, the deflected extensions 136 straighten, forcing the tapered portions 138 into the circumferential groove 160. The clinician experiences tactile feedback, such as a snap-like sensation, that indicates that the explant plug device 300 is coupled to the ventricular cuff 120. The bottom surface 125 of the linking member 124 engages the circumferential flange 162 of the explant plug device 300.

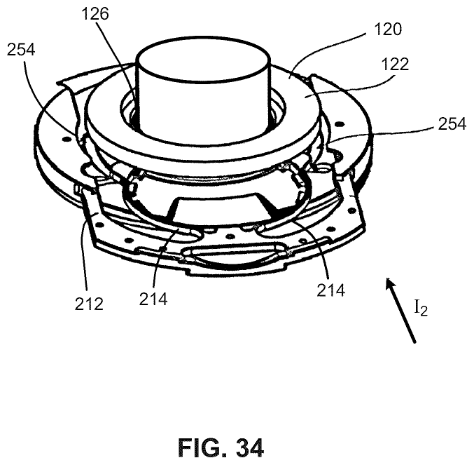

Referring to FIG. 34, the clinician advances the clip 200 into the cap assembly 302. The guide rails 212 of the clip 200 travel in the slots 252, guiding the clip 200 as it travels linearly in a plane above the top side 256, in the direction of arrow I₂. As the clip 200 travels relative to the cap assembly 302, the arms 214 flex laterally due to engagement of the posts 219 with the angled walls 257, 258 defining the channels 254. The arms 214 move laterally outward to admit the ventricular cuff 120 and then laterally inward to engage the ventricular cuff 120.

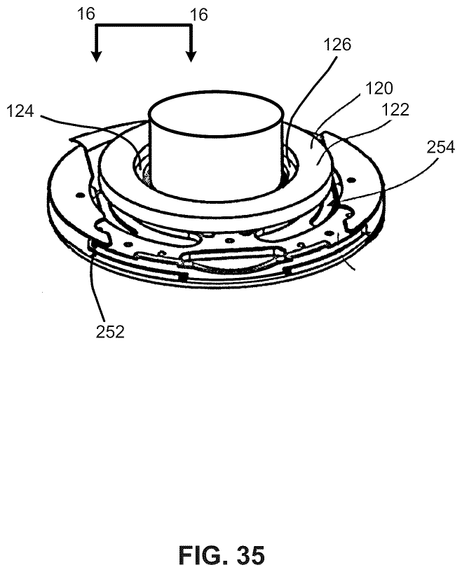

Referring to FIG. 35, the clip 200, in its locked position, limits travel of the explant plug device 300 relative to the ventricular cuff 120. The engagement of the posts 219 with the angled walls 257, 258 that define the channels 254 forces the arms 214 inward such that the teeth 216 of the arms 214 are disposed under the outwardly extending flange of the ventricular cuff 120. The outwardly extending flange is captured between the teeth 216 and the cap assembly 302. The engagement of the teeth 216 to the outwardly extending flange presses the outwardly extending flange against the cap assembly 302, forming a seal (shown in FIG. 36).

In an implanted state, after the clip 200 is in its locked position, the explant plug device 300 is securely retained to the ventricular cuff 120. While the clip 200 is in its locked position, an extremely large force is required to remove the explant plug device 300 from the ventricular cuff 120. For example, the force required to forcibly separate the explant plug device 300 from the ventricular cuff 120 while the clip 200 is in its locked position can be as large as the force required to tear the ventricular cuff 120 from the heart 16.

The explant plug device 300 can include the blood flow channel seal assembly 44 described herein with respect to the explant plug device 40. For example, the blood flow channel seal assembly 44 can be configured to extend at least partially through the blood flow channel 130 of the ventricular cuff 120. The blood flow channel seal assembly 44 can be configured to be detachably mounted to the cap assembly 302. The blood flow channel seal assembly 44 can include the O-ring seal 48 configured to interface with an inner surface of the ventricular cuff 120 that defines the blood flow channel 130. The blood flow channel seal assembly 44 can be configured for filling the cored opening 22 in the heart 16. The blood flow channel seal assembly 44 can be configured to extend into the ventricle. The blood flow channel seal assembly 44 can be the adjustable blood flow channel assembly 44-ADJ shown in FIG. 13.

The explant plug device 300 can have any suitable configuration. For example, the ventricular cuff 120 can include an outward facing circumferential groove and ridges disposed at least partially within the outward facing circumferential groove. The clip 200 can include two arms 214 that are engaged with the at least one of the ridges when the clip 200 is ins the locked position. The blood flow channel 130 of the ventricular cuff 120 can have a longitudinal axis. The clip 200 can be configured to be moved transverse to the longitudinal axis, relative to the cap assembly 302, to move the clip 200 from the unlocked position to the locked position. The clip 200 can be configured to be moved perpendicular to the longitudinal axis, relative to the cap assembly 302, to move the clip 200 from the unlocked position to the locked position. The latching mechanism 304 can define channels along which the two arms 214 travel during movement of the clip 200 from the unlocked position to the locked position. The channels can define detents configured to impede movement of the clip 200 into the locked position when the explant plug device 300 is not mounted to the ventricular cuff. Each of the two arms 214 can engage one of the detents independent of whether the other of the two arms 214 engages one of the detents. The detents and the arms 214 can be configured so that engagement of either of the two arms 214 with one of the detents impedes the clip 200 from reaching the locked position. The detents and the arms 214 can be configured so that when the clip 200 is moved toward the locked position from the unlocked position and the cap assembly 302 is mounted to the ventricular cuff 120, the ventricular cuff 14 blocks engagement of the two arms 214 with the detents. The two arms 214 can include teeth that are engaged with at least some of the ridges when the clip 200 is in the locked position. The teeth can be configured to engage the ventricular cuff 120 when the clip 200 is in the locked position to force the cap assembly 302 into a fully seated position against the ventricular cuff 120. The ridges can be disposed on the outwardly extending flange of the ventricular cuff 120. Each of the teeth can have a chamfered edge that engages the outwardly extending flange to force the cap assembly 302 into the fully seated position against the ventricular cuff 120. The clip 200 can include a visual indicator The visual indicator can be exposed when the clip 200 is not in the locked position. The visual indicator can be obscured when the clip 200 is in the locked position. The clip 200 can include a latch that impedes movement of the clip 200 from the locked position. The explant plug device 300 can include a sealing ring configured to engage with an inward facing surface of the ventricular cuff 120 when the explant plug device 300 is mounted to the ventricular cuff 120.

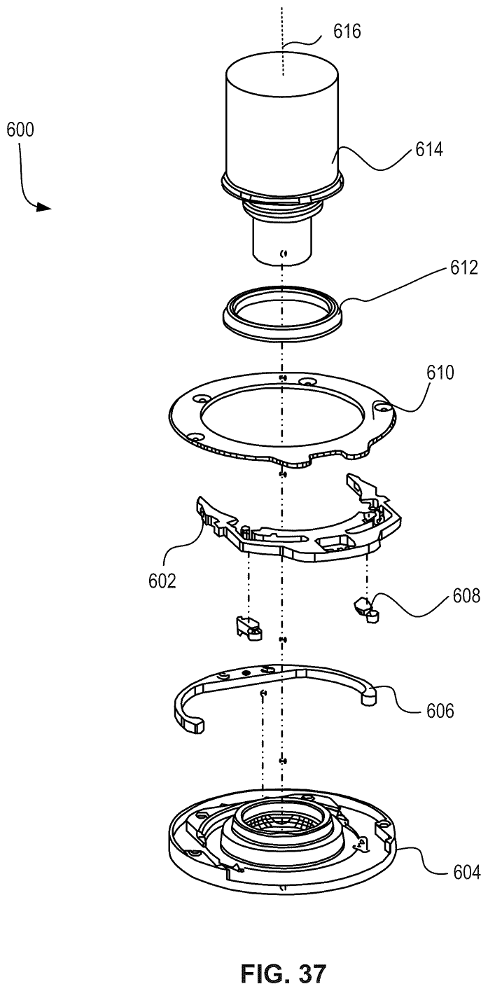

FIG. 37 is an exploded view of an explant plug device 600. The description of various systems as described with respect to the explant plug device 300 is relevant relative to the explant plug device 600. Accordingly, similar components have similar numbering, form, and function unless otherwise noted herein. The explant plug device 600 includes a retention clip 602, a base member 604, a spring arm member 606, pivoting components 608, a retention clip cover 610, a seal 612, and a blood flow channel seal assembly 614, which extends along a longitudinal axis 616. The base member 604 has recesses configured to accommodate the retention clip 602. The spring arm member 606 and the retention clip cover 610 are mounted to the base member 604. The retention clip cover 610 and the base member 604 form a housing into which the retention clip 602 extends. The retention clip 602 is translatable relative to the base member 604 between a retracted configuration (configured to accommodate mounting of the explant plug device 600 to ventricular cuff 120) and a retention configuration (configured to retain the explant plug device 600 to the ventricular cuff 120). The blood flow channel assembly 614 is configured for insertion into the blood flow channel 130 of the ventricular cuff 120. The pivoting components 608 are configured to be in an insertion orientation that accommodates insertion of the retention clip 602 to the retention configuration when the explant plug device 600 is properly seated on the ventricular cuff 120. The pivoting components 608 are configured to be in blocking orientation that blocks insertion of the retention clip 602 to the retention configuration when the explant plug device 600 is not properly seated on the ventricular cuff 120.

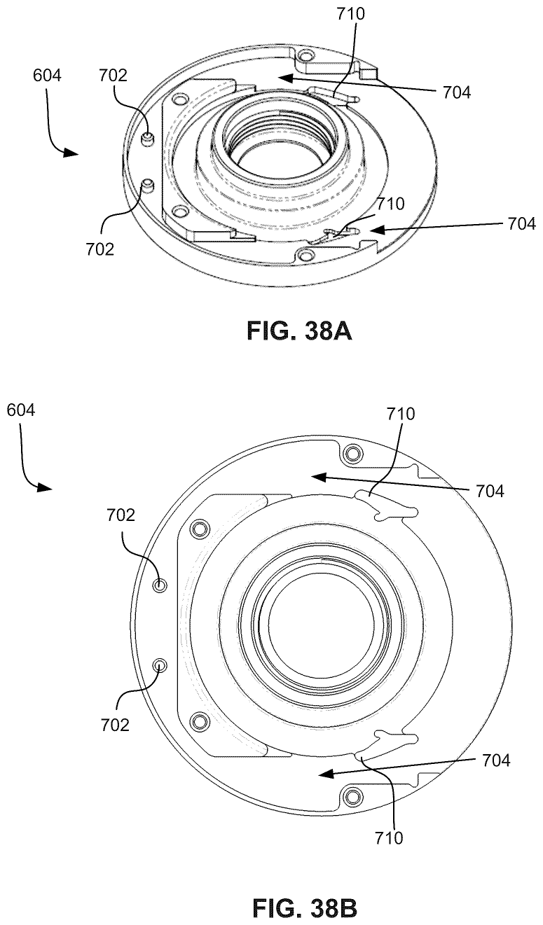

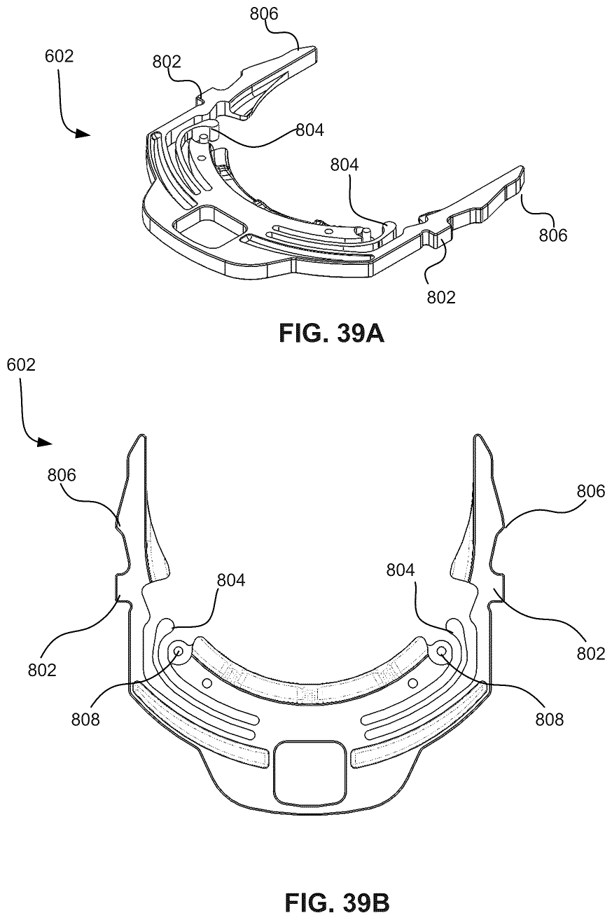

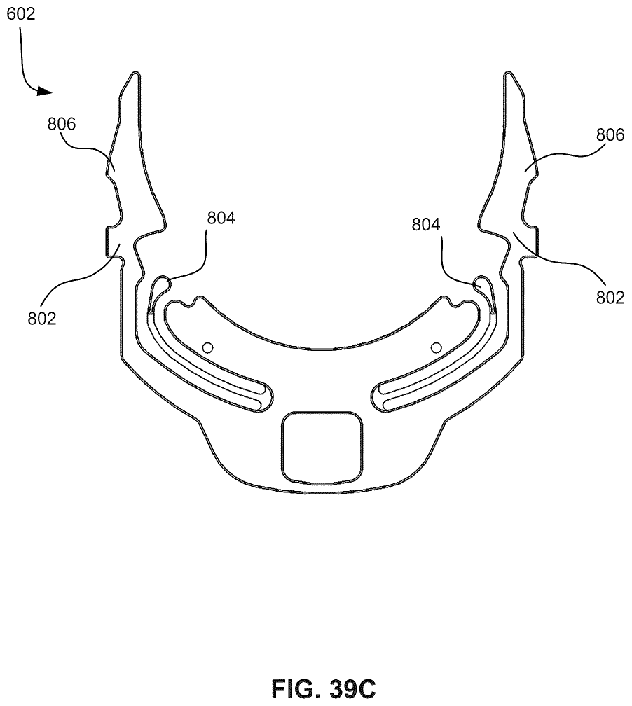

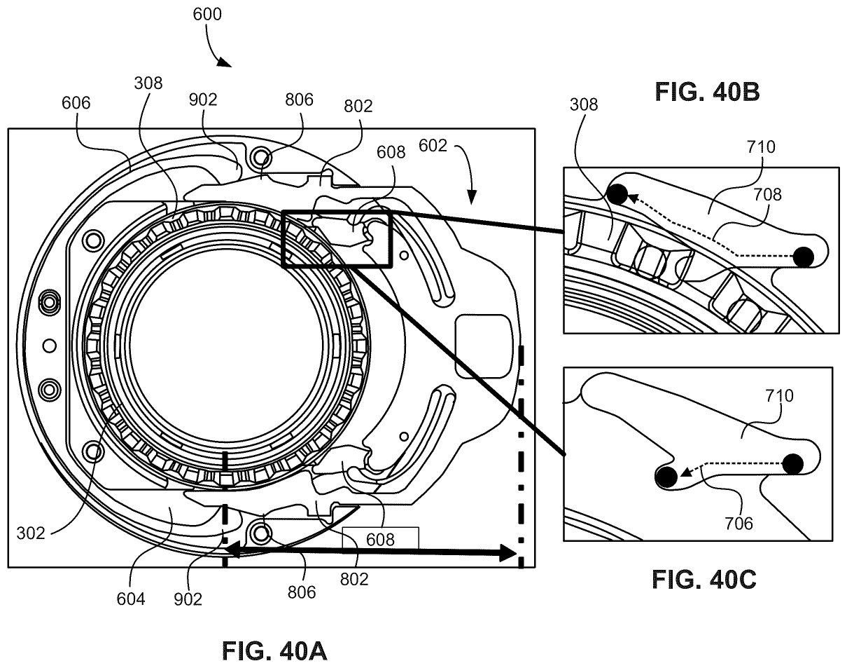

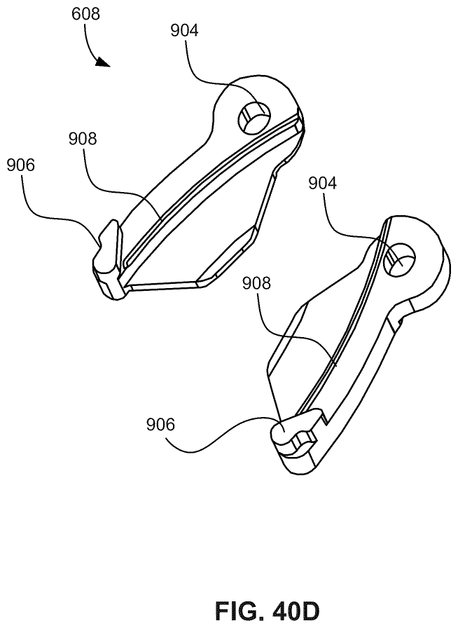

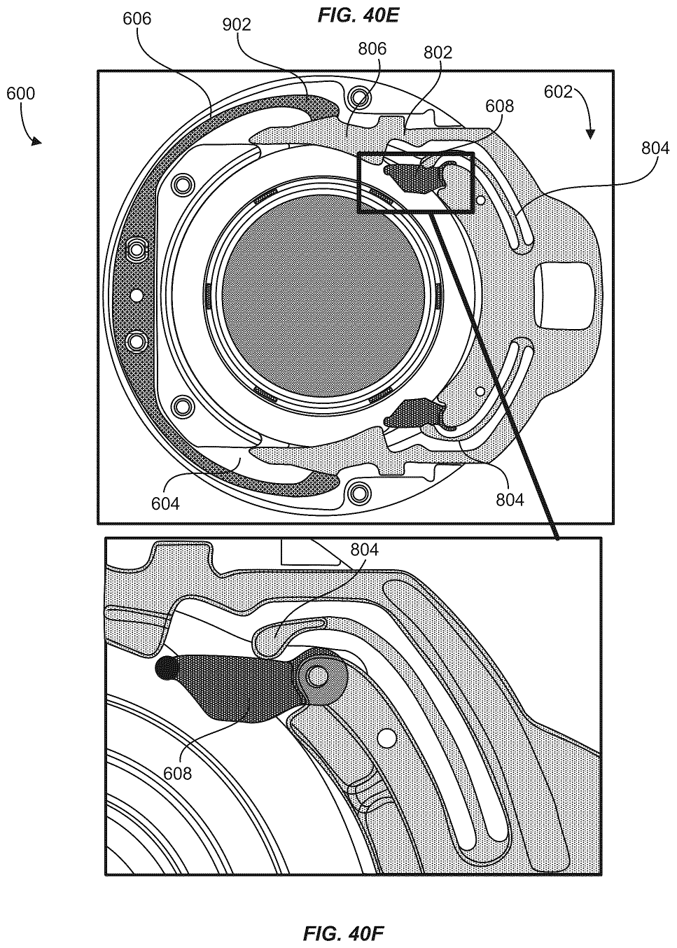

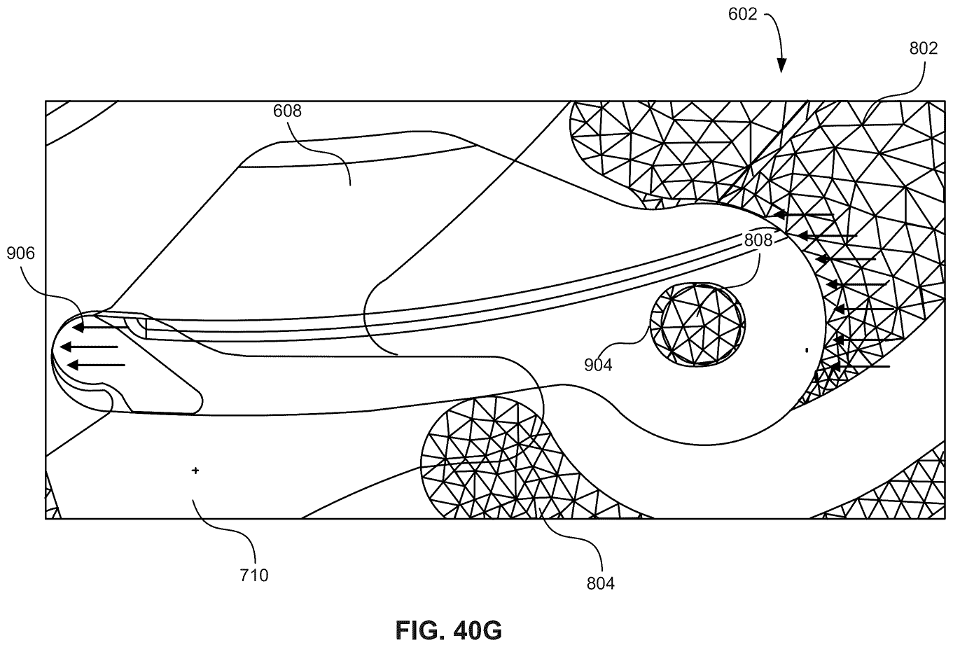

FIG. 38A is an oblique view of the base member 604. FIG. 38B is a top view of the base member 604. The base member 604 includes protrusions 702 for securing the spring arm member 606 in position. The base member 604 defines retention clip passages 704. Retention clip arms 802 of retention clip 602 are slidably disposed within the retention clip passages 704. When the explant plug device 600 is properly seated onto the ventricular cuff 120, the pivoting components 608 are oriented to accommodate full insertion of the retention clip 602 into the retention clip passages 704 to the retention configuration. When the explant plug device 600 is not properly seated onto the ventricular cuff 120, the pivoting components 608 are oriented to block full insertion of the retention clip 602 into the retention clip passages 704 to the retention configuration. The base member 604 further defines recesses 710. Each of the pivoting components 608 includes a pivot 904 (as shown in FIG. 40D) that are captured by a respective post 808 of the retention clip 602 (described with respect to FIG. 39A, FIG. 39B, and FIG. 39C below). Each of the pivoting components include a protrusion 906 (shown in FIG. 40D) that extends into one of the recesses 710. Each of the recesses 710 accommodates a lockout path 706 of the protrusion 906 and a non-lockout path 708 of the protrusion 906. When the explant plug device 600 is not properly seated onto the ventricular cuff 120, each of the pivoting components 608 is oriented to traverse the lockout path 706 so that the protrusion 906 comes into contact with the wall of the recess 710 at the end of the lockout path, thereby blocking further insertion of the retention clip 602 into the retention clip passages 704. When the explant plug device 600 is properly seated onto the ventricular cuff 120, each of the pivoting components 608 is oriented to traverse the non-lockout path 708, which is shaped to accommodate full insertion of the retention clip 602 into the retention clip passages 704 to the retention configuration of the retention clip 602.

FIG. 39A is an oblique view of a retention clip 602. FIG. 39B is a bottom view of the retention clip 602. FIG. 39C is a top view of the retention clip 602. The retention clip 602 is repositionable relative to the base member 604 between a retracted position and an engaged position, which corresponds to a retention configuration of the explant plug device 600. The retention clip 602 in the retracted position accommodates insertion of the blood flow channel seal assembly 614 into the blood flow channel 130 of the ventricular cuff 120 to mount the explant plug device 600 onto the ventricular cuff 120 into a mounted configuration (e.g., a properly seated configuration).

The retention clip 602 includes retention clip arms 802 that are slidably disposed within the retention clip passages 704 defined by the base member 604. The retention clip arms 802 are configured to interface with the outward facing circumferential flange of the ventricular cuff 120 to secure the explant plug device 600 to the ventricular cuff 120. The retention clip 602 includes spring arms 804 that interact with the pivoting members 608. According to at least some embodiments, the spring arms 804 are thicker at the free end to prevent twisting in the retention clip passages 704 while flexing. The thickness and height of the spring arms 804 can be tuned to adjust the amount of lateral force applied to the pivoting members 608. The retention clip arms 802 can include one or more ridges 806 for engaging with and securing the retention clip arms 802 relative to the spring arm member 606 within the base member 604. As shown in FIG. 39B, the bottom surface of the retention clip 602 includes posts 808 that couple to the pivoting components 608.

FIG. 40A shows retention related components of the explant plug device 600. FIG. 40B shows one of the coupling paths 708 for a respective one of the protrusions 906. FIG. 40C shows one of the lockout paths 706 for the respective one of the protrusions 906. FIG. 40D shows a bottom view of the pivoting components 608. FIG. 40E shows the retention related components of the explant plug device 600 in a lockout configuration. FIG. 40F shows a close-up view of a lockout related portion of the retention related components of the explant plug device 600. FIG. 40G illustrates compression forces reacted by each of the pivoting components 608. As shown in FIG. 40A, the spring arm member 606 includes flexible arms 902 configured to engage the retention clip arms 802. The flexible arms 902 are configured to wrap around and secure the ridges 806 of the retention clip arms 802 in a locked position and inhibit repositioning of the retention clip 602 relative to the base member 604.

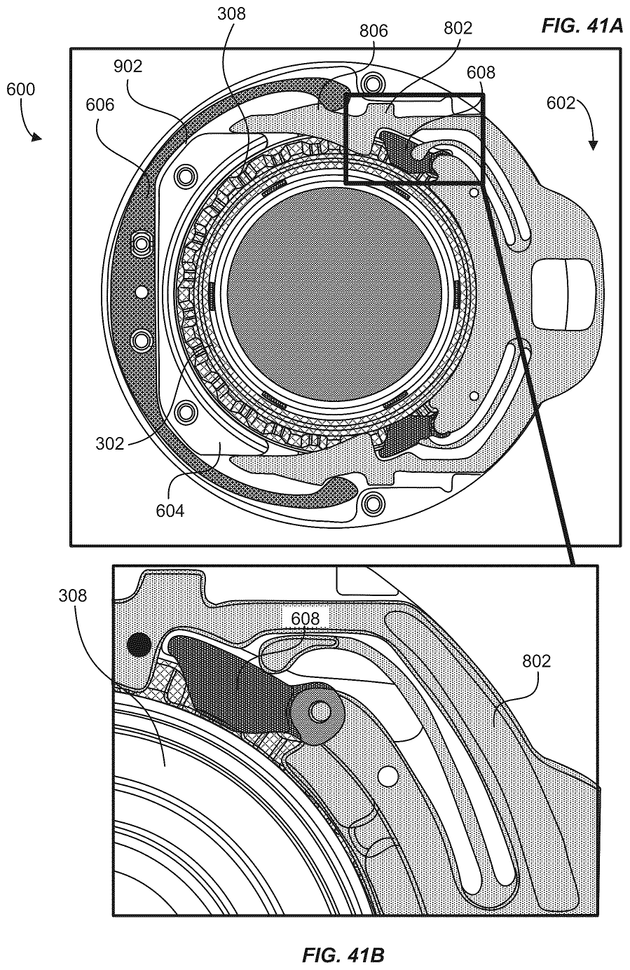

FIG. 40A shows the retention clip 602 in a retracted position and the ventricular cuff 120 positioned such that the ventricular cuff flange 308 is seated relative to the explant plug device 600. In this position, the ventricular cuff flange 308 abuts against the pivoting components 608 and, as the retention clip 602 is advanced toward the locked position, the ventricular flange 308 displaces the pivoting components 608 radially outward to the positions shown in FIG. 41A and FIG. 41B. FIG. 40B further illustrates how the presence of the ventricular cuff flange 308 positions the protrusions 906 of the pivoting components 608 such that the protrusions 906 travel along the non-lockout paths 708, thereby accommodating full insertion of the retention clip 602 to produce the retention configuration of the explant plug device 600. In contrast, when the ventricular cuff flange 308 is not present (e.g., when the explant plug device 600 is not properly seated onto the ventricular cuff 120), the protrusions 906 of the pivoting components 608 travel along the lockout paths 706 so that the protrusions 906 come into contact with end walls of the recesses 710 at the end of the lockout paths 706 and thereby block further insertion of the insertion clip 602 relative to the base member 602.

As shown in FIG. 40D, each of the pivoting components 608 includes a protrusion 906. The posts 808 of the retention clip 602 insert into the pivot journals 904. Each of the pivoting components 608 includes a protrusion 906 that slides along the detents 710 of the base member 604.

As shown in FIG. 40E, when the ventricular cuff flange 308 is not present, the spring arms 804 keep the protrusions 906 of the pivoting components 608 on the lockout tracks 706 (shown in FIG. 40C). FIG. 40G illustrates compression forces reacted by each of the pivoting components 608 in the lockout configuration. The geometry of the pivoting components 608 is designed to withstand the compressive forces generated via a surgeon trying to insert the retention clip 602 when the explant plug device 600 is not sufficiently seated onto the ventricular cuff 120. The ability of the pivoting components 608 to react such compressive forces is especially important since the surgeon may forcefully push the retention clip 602 radially in toward the center of the explant plug device 600 expecting the retention clip 602 to lock into place. Each of the pivoting components 608 is configured to react such compressive force via external end bearing surfaces in contact with the retention clip 602 and the base member 604 respectively. The pivot slot 904 and the clearance between the end surface of the pivoting component 608 are sized so that the compressive force is transferred via the end bearing surface instead of via the pivot slot 904 to protect the post 808 from damage. For example, in various embodiments, the length of the pivot slot 904 is long enough along the direction of compression to avoid applying force to 808 in the lockout configuration.

FIG. 41A shows a top view of the retention related components of the explant plug device 600 in a retention configuration of the explant plug device 600. FIG. 41B shows a close-up view of the retention related components of the explant plug device 600 in the retention configuration. As shown in FIG. 41A, when the explant plug device 600 is properly seated onto the ventricular cuff 120, the ventricular cuff flange 308 displaces the pivoting components 608 outwardly thereby accommodating full insertion of the retention clip 602 into the base member 604 such that the flexible arms 902 of the spring arm member 606 extend around the ridges 806 of the retention clip arms 802 and secure the retention clip 602 in the locked position. In various embodiments, the retention clip 602 is not repositionable once the retention clip 602 is in the locked position. In the illustrated embodiment, the steep backside angle of the ridge 806 prevents the retention clip 602 from being retracted without the use of an additional tool (not shown).

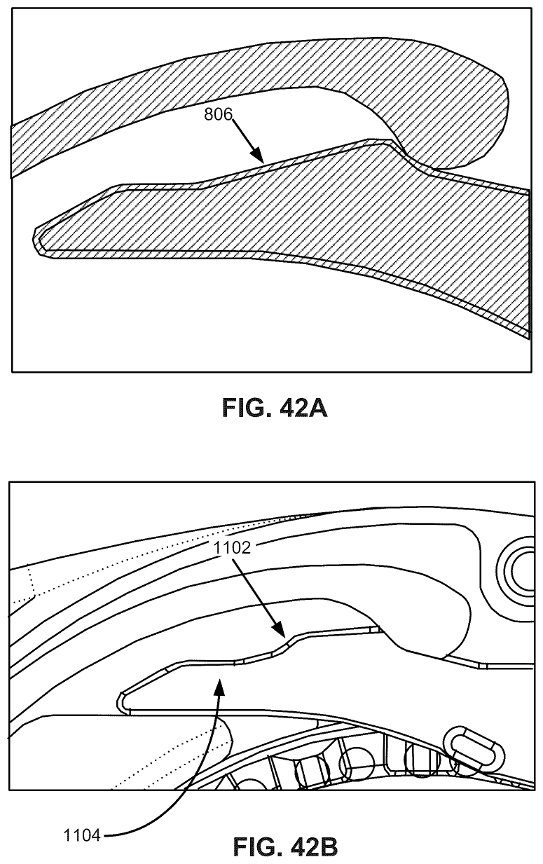

FIG. 42A and FIG. 42B show a close-up views of two different embodiments of end portions of the retention clip 602 that interface with the spring arm member 606. FIG. 42A illustrates the retention clip ridge 806 as shown in previous figures. FIG. 42B illustrates an alternative retention clip ridge 1102 having a sharp jog 1104 that provides more pronounced tactile feedback to indicate that the retention clip 602 has entered the retracted position. The alternative retention clip ridge 1102 may be used in any of the embodiments of a retention clip 602 described herein. The alternative retention clip ridge 1102 may also reduce the occurrence of inadvertently pushing the retention clip 602 from the retracted position to the locked or lockout position by increasing the initial force required to push the retention clip 602 in radially. According to various embodiments, the slope and contours of the profile can be adjusted beyond what is shown in FIG. 42A and FIG. 42B to fine tune the forces encountered when actuating the retention clip 602 between retracted and locked/lockout positions (bidirectionally).

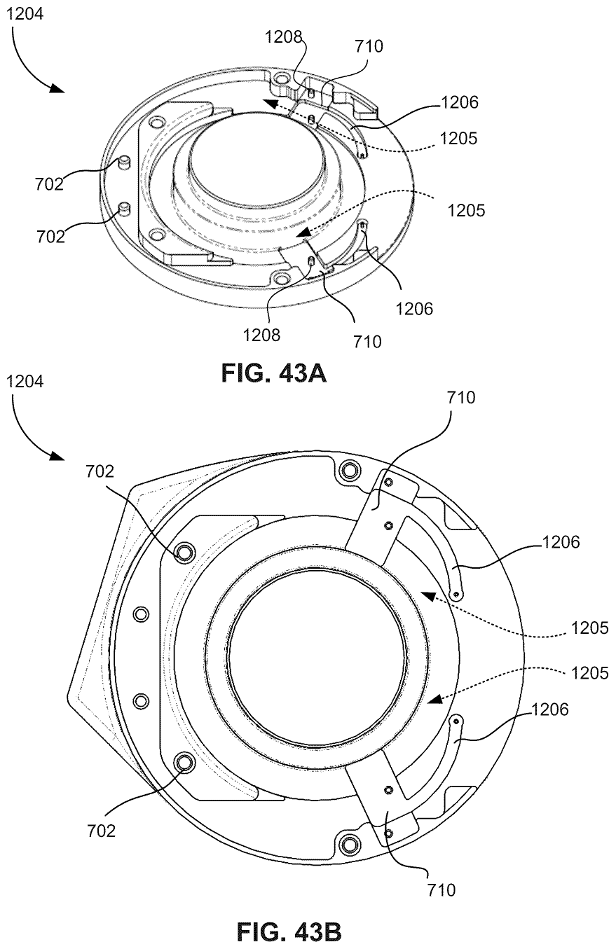

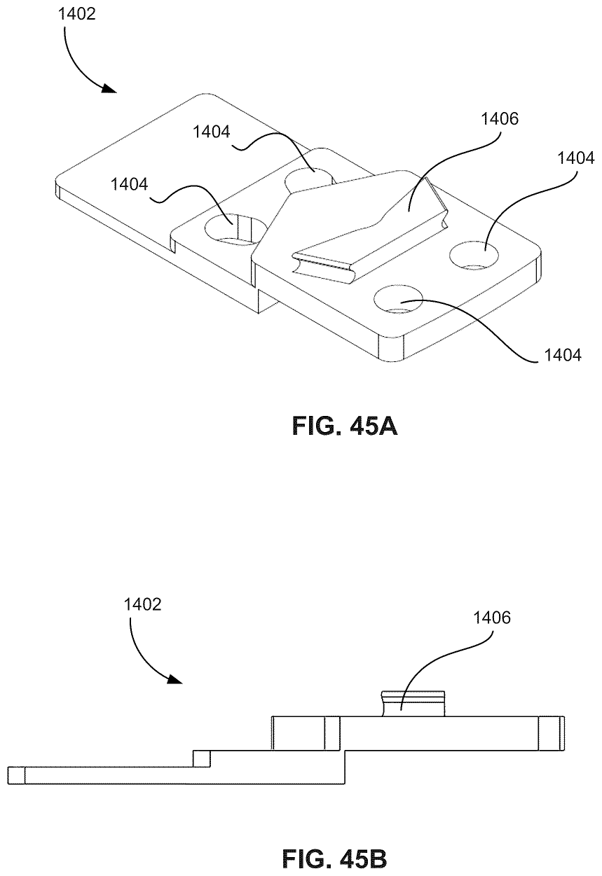

FIG. 43A is an oblique view of a base member 1204 of an explant plug device 1200. The description of various systems as described with respect to the explant plug devices 300, 600 is relevant relative to the explant plug device 1200. Accordingly, similar components have similar numbering, form, and function unless otherwise noted herein. that is configured similar to the explant plug device 300. FIG. 43B is a top view of the base member 1204. The base member 1204 includes protrusions 702 for securing the spring arm member 606 in position. The base member 1204 defines retention clip passages 1205. Retention clip arms 802 of a retention clip 1202 are slidably disposed within the retention clip passages 1205. The base member 1204 further defines recesses 710 that accommodate spring-loaded stop components 1402 (shown in FIG. 45A and FIG. 45B) that are spring biased towards a retention clip blocking position and displaced to a retention clip insertion accepting position via proper seating of the explant plug device 1200 onto the ventricular cuff 120. Each of the recesses 710 defines at least one post 1208 that is slidingly received in a respective aperture 1404 in the spring-loaded stop component 1402. Each of the stop components 1402 includes a stop ledge 1406 that blocks full insertion of the retention clip 1202 when the stop component 1402 is in the retention clip blocking position. The base member 1204 includes slots 1206 for springs that bias the stop components 1402 towards the retention clip blocking position and deform to accommodate displacement of the stop components 1402 from the retention clip blocking position to the retention clip insertion accepting position.

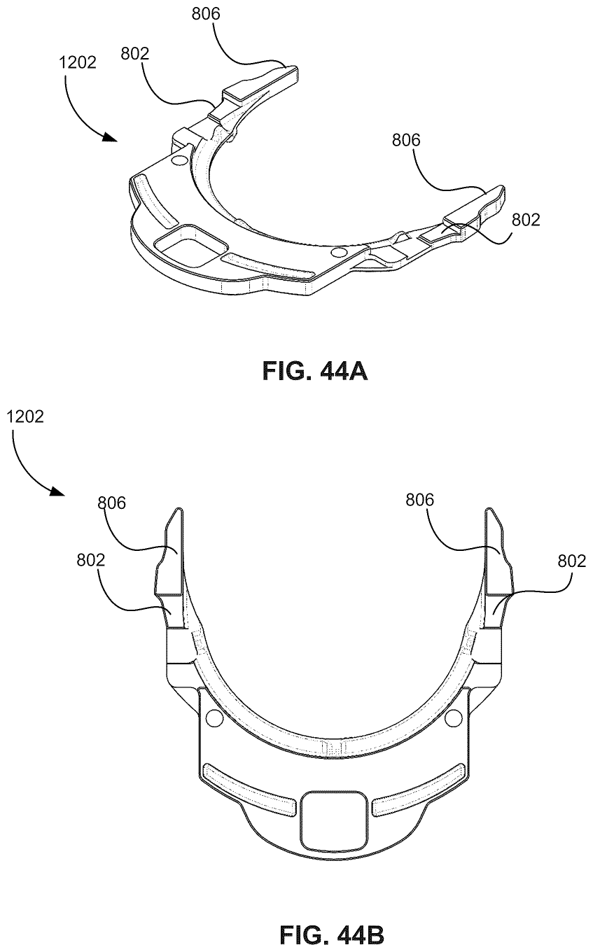

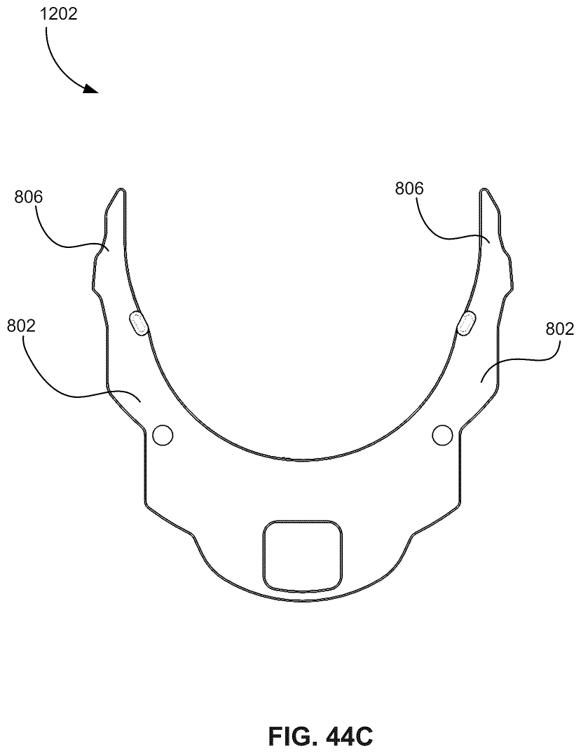

FIG. 44A is an oblique view of a retention clip 1202 of the explant plug device 1200. FIG. 44B is a bottom view of the retention clip 1202. FIG. 44C is a top view of the retention clip of FIG. 44A. The retention clip 1202 is repositionable relative to the base member 1204 between a retracted position and an engaged (e.g., locked) position. In the retracted position, the retention clip 1202 accommodates insertion of the blood flow channel seal assembly 306 into the blood flow passage 130 of the ventricular cuff 120 to mount the explant plug device 1200 onto the ventricular cuff 120.

The retention clip 1202 includes retention clip arms 802 that are slidably disposed within retention clip passages 1205. The retention clip arms 802 are configured to engage the circumferential flange 308 of the ventricular cuff 120 to retain the explant plug device 1200 to the ventricular cuff 120. The retention clip arms 802 include one or more ridges 806 for engaging with and securing the retention clip arms 802 relative to the spring arm member 606 within the base member 1204 similarly to the retention clip 602.

FIG. 45A is an oblique view of the stop component 1402. FIG. 45B is a side view of the stop component 1402. Each stop component 1402 interacts with a curved spring positioned in the slots 1206 of the base member 1204. The apertures 1404 of the stop components 1402 are held captive by posts 1208 machined into the base member 1204.

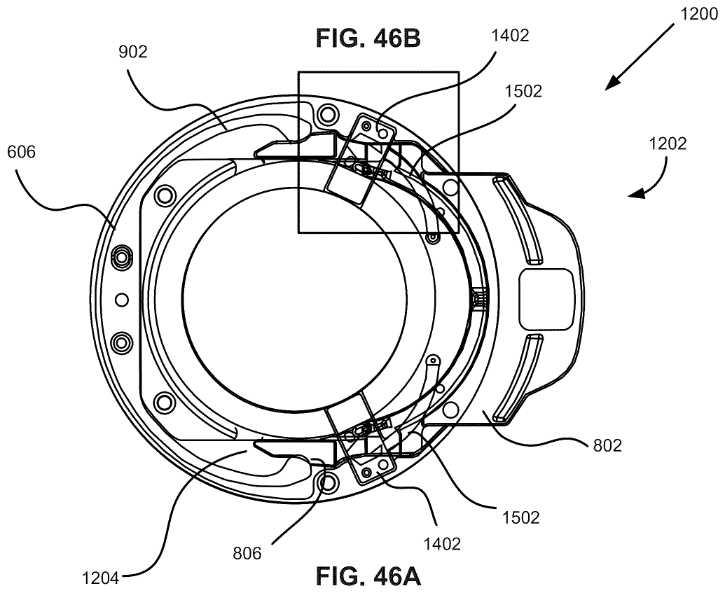

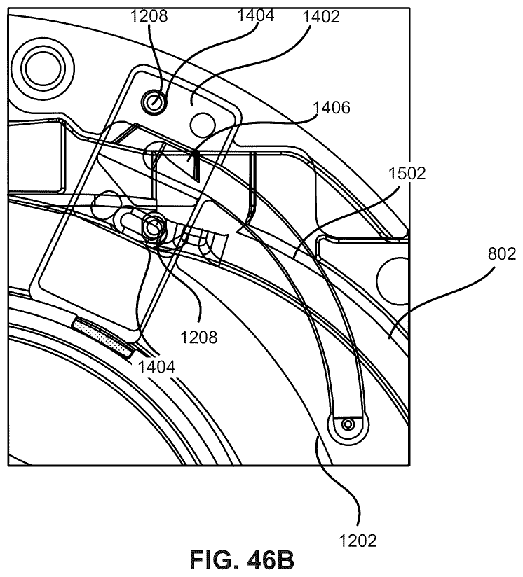

FIG. 46A is a top view of retention related components of the explant plug device 1200. FIG. 46B is a close-up view of lockout-related components of the retention related components of the explant plug device 1200. As shown in FIG. 46A, the spring arm member 606 includes flexible arms 902 configured to engage the retention clip arms 802 of the retention clip 1202. In particular, the flexible arms 902 of the spring arm member 606 are configured to wrap around and secure the one or more ridges 806 of the retention clip 802 in a locked position and inhibit repositioning of the retention clip arms 802 relative to the base member 1204. FIG. 46A shows the retention clip 1202 is a retracted position. The base member 1204 includes springs 1502. The springs 1502 can be of various shapes and materials, fine-tuned to apply enough vertical force to ensure the stop components 1402 are biased into the retention clip blocking position when the retention clip 1202 is in the retracted and lockout positions. The springs 1502 can have a suitable spring rate that accommodates deformation of the springs 1502 without causing a significant increase in the amount of force required to fully seat the explant plug device 1200 onto the ventricular cuff 120. In some embodiments, an elastomeric material is disposed underneath the stop component to act as a spring that biases the stop component 1402 toward the retention clip insertion blocking position. The posts 1208 can be machined into the base member 1204.

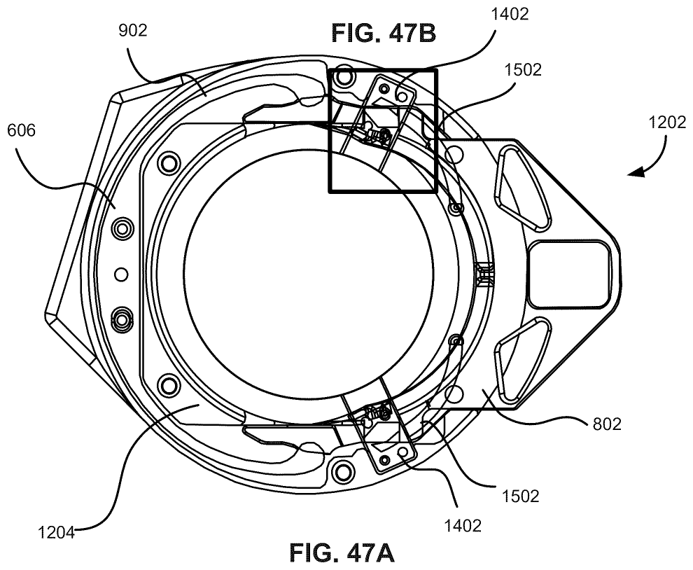

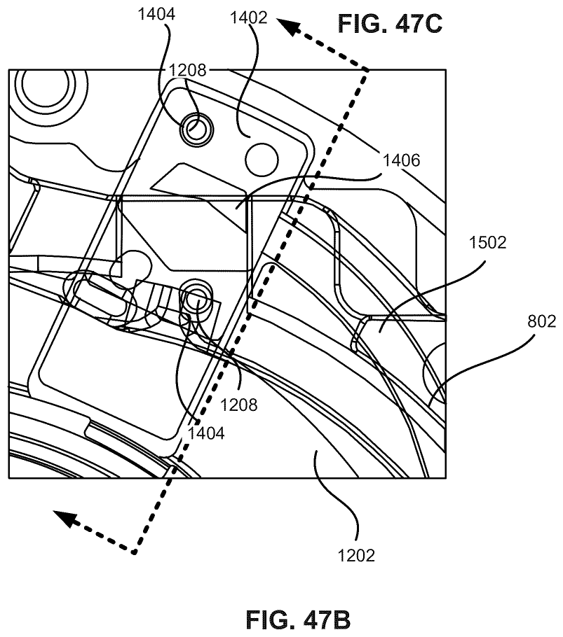

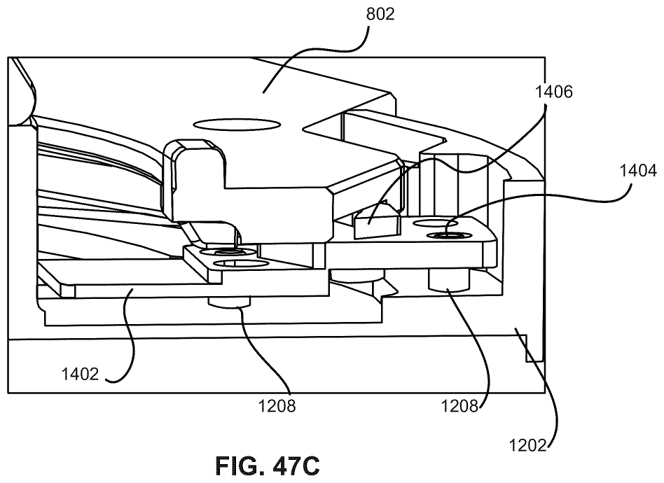

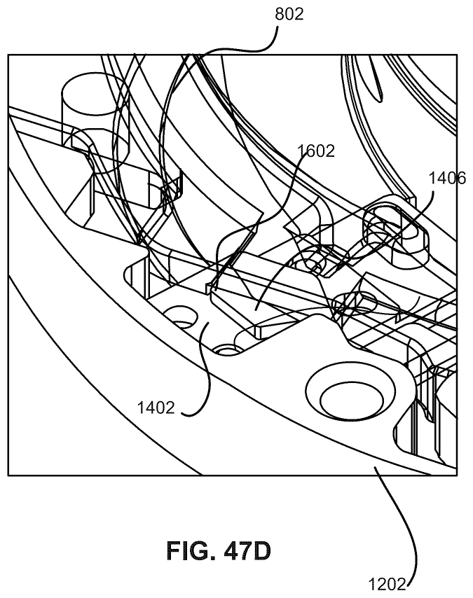

FIG. 47A shows the retention related components of the explant plug device 1200 in the lockout configuration. FIG. 47B, FIG. 47C, and FIG. 47D show lockout-related components of the explant plug device 1200 in the lockout configuration. The springs 1502 contact the underside of the stop components 1402 and push the stop components 1402 up vertically along the posts 1208 machined into the base member 1204, thereby aligning a stop ledge 1406 with an interfacing ledge of the retention clip 1202. When the explant plug device 1200 is not seated onto the ventricular cuff 120, the stop components 1402 remain elevated by the springs 1502 and the stop ledges 1406 prevent the retention clip 1202 from advancing to the point where the retention clip 1202 locks with the spring arm member 606. The elevated stop components 1402 result in a lockout configuration as shown in FIG. 47C. The vertical wall of the stop ledge 1406 ledge stops forward movement of the retention clip 1202, resulting in the lockout configuration.

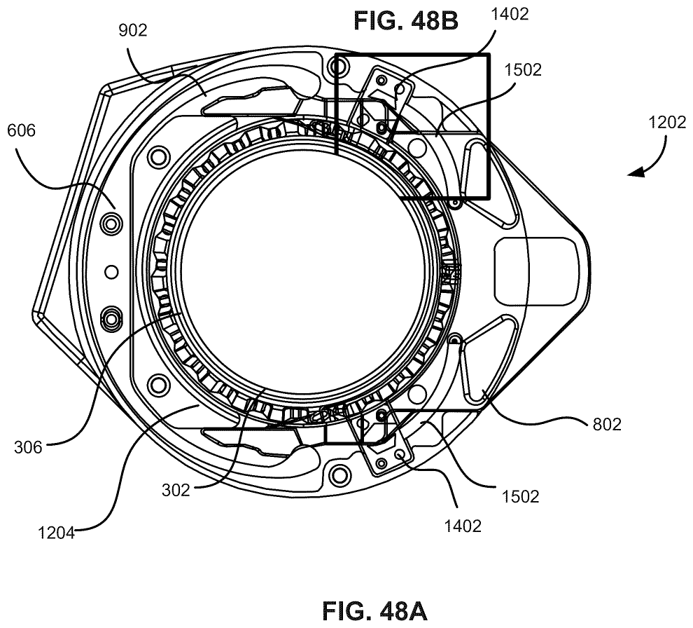

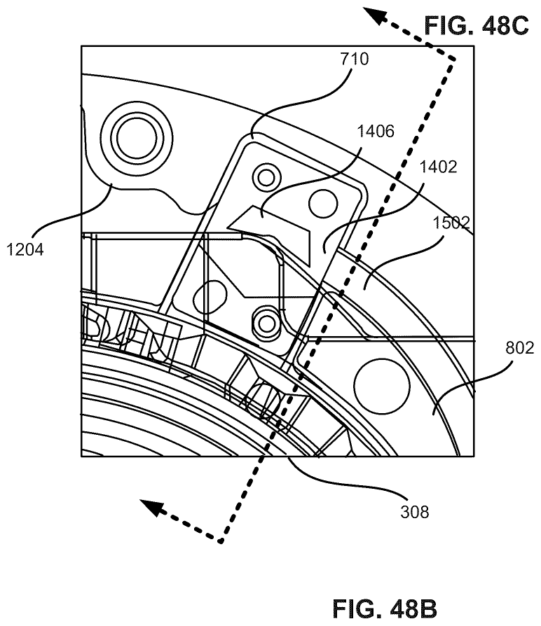

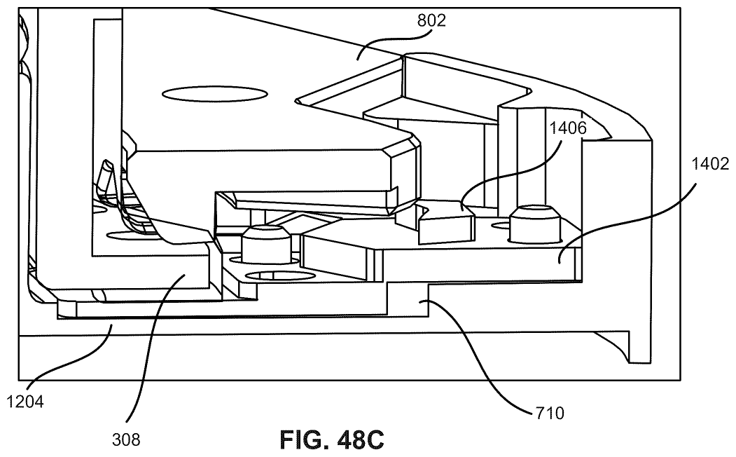

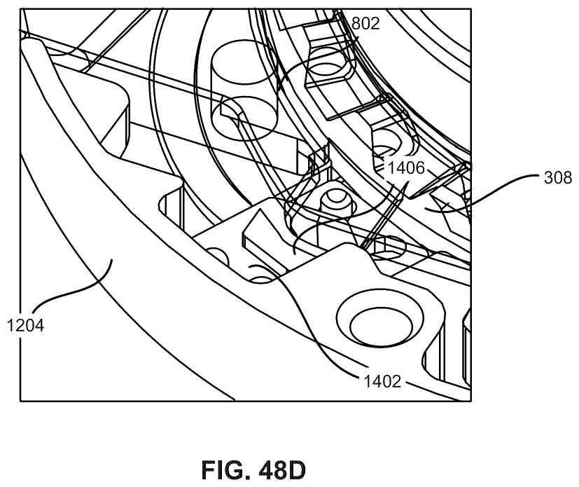

FIG. 48A is a top view of the retention related components of the explant plug device 1200 in the retention configuration. FIG. 48B, FIG. 48C, and FIG. 48D show views of lockout-related components of FIG. 48A in the retention configuration. When the explant plug device 1200 is properly seated onto the ventricular cuff 120, the circumferential flange 308 of the ventricular cuff 120 depresses the stop components 1402 into the recesses 710 of the base member 1204. The depression of the stop components 1402 clears the way for full insertion of the retention clip 1202 to the position where the spring arm member 606 deflects past the retention clip arm 802 ridges 806, thereby locking the retention clip 1202 into place.

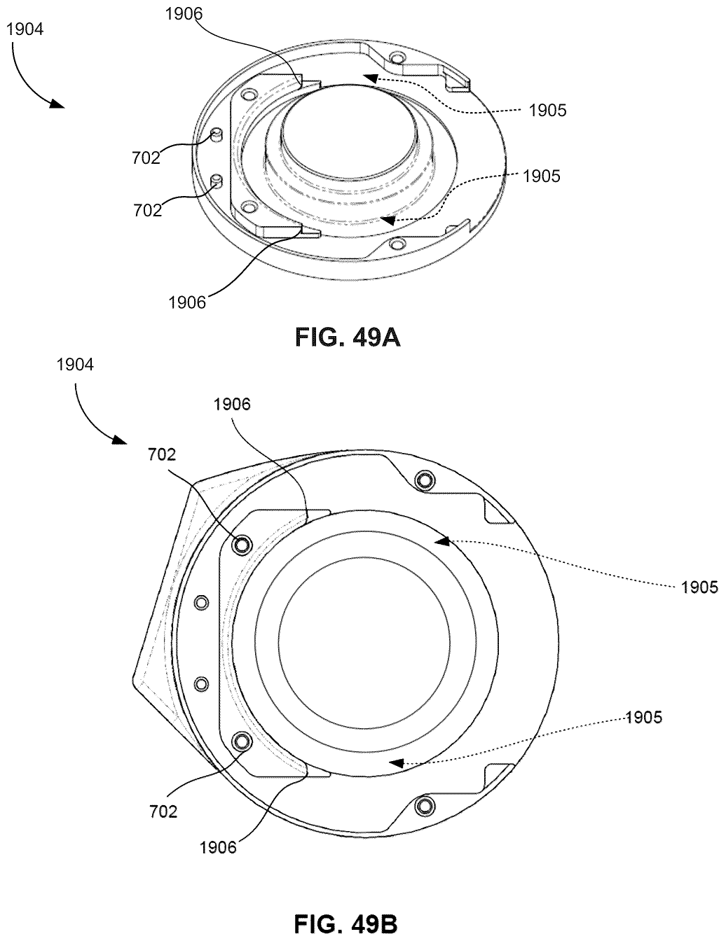

FIG. 49A is an oblique view of a base member 1904 of an explant plug device 1900. The description of various systems as described with respect to the explant plug devices 300, 600, 1200 is relevant relative to the explant plug device 1900. Accordingly, similar components have similar numbering, form, and function unless otherwise noted herein. FIG. 49B is a top view of the base member 1904. The base member 1904 includes protrusions 702 for securing the spring arm member 606 in position. The base member 1904 defines retention clip passages 1905. Retention clip arms 802 of cuff retention clip 1902 are slidably disposed within the retention clip passages 1905. The retention clip 1902 includes two-part cam components 2002, which block full insertion of the retention clip 1902 when the explant plug device 1900 is not properly seated onto the ventricular cuff 120 and accommodates full insertion of the retention clip 1902 when the explant plug device 1900 is properly seated onto the ventricular cuff 120. The base member 1904 further defines stop surfaces 1906 that direct the two-part cam components 2002 between a first position and a second position. The first position of the two-part cam components 2002 corresponds to a retention configuration of the retention clip 1902. The second position of the two-part cam components 2002 corresponds to the lockout position or configuration of the retention clip 1902.

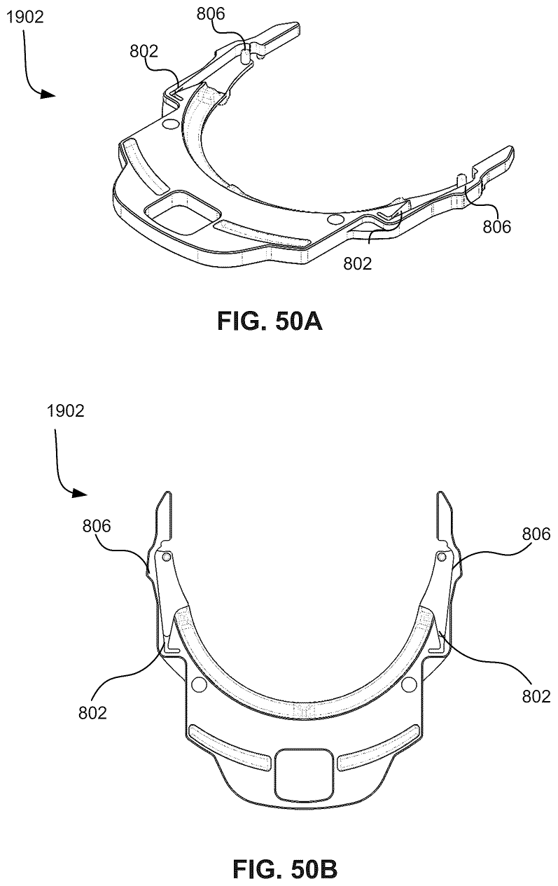

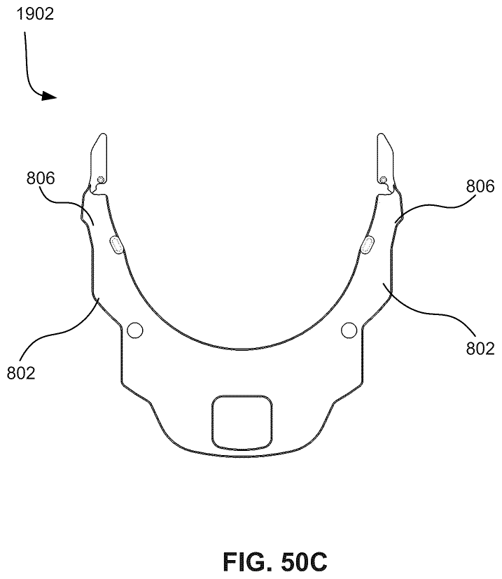

FIG. 50A is an oblique view of the retention clip 1902 of the explant plug device 1900. FIG. 50B is a bottom view of the retention clip 1902. FIG. 50C is a top view of the retention clip 1902. The retention clip 1902 is repositionable relative to the base member 1904 between a retracted position and an engaged (e.g., locked) position. In the retracted position, the retention clip 1902 accommodates insertion of the blood flow channel seal assembly 306 into the blood flow passage 130 of the ventricular cuff 120 to mount the explant plug device 1200 onto the ventricular cuff 120.

The retention clip 1902 includes retention clip arms 802 that are slidably disposed within retention clip passages 1905 defined by the base member 1904. The retention clip arms 802 are configured to engage the circumferential flange 308 of the ventricular cuff 120 to secure the explant plug device 1900 to the ventricular cuff 120. The cuff lock arms 802 can include one or more ridges 806 for engaging with and securing the retention clip arms 802 relative to the spring arm member 606 mounted to the base member 1904.

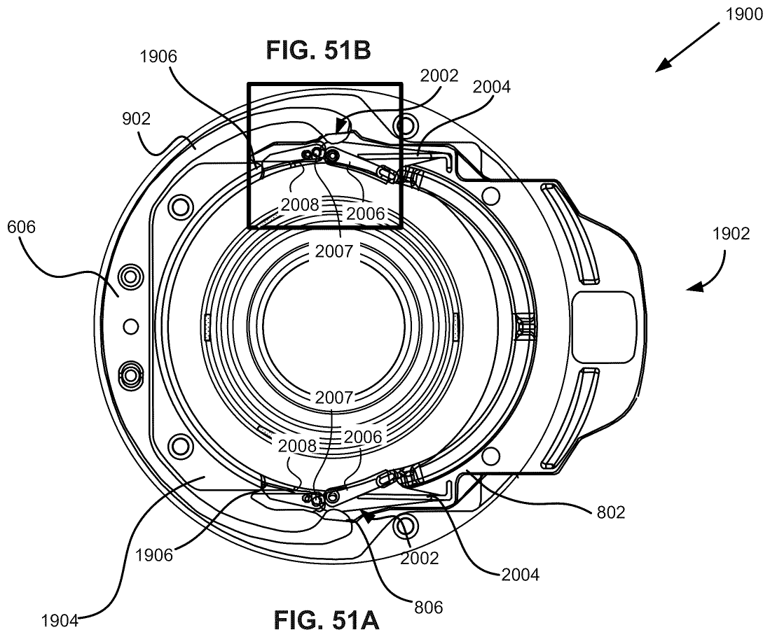

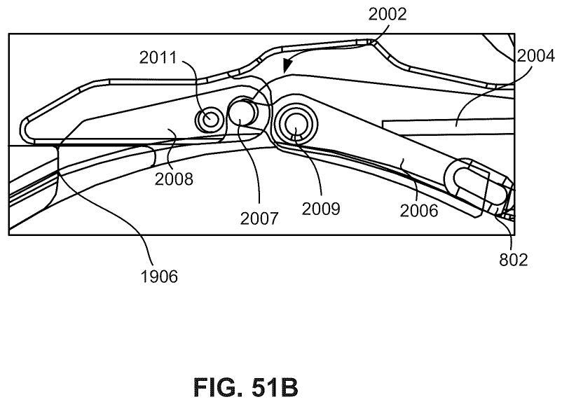

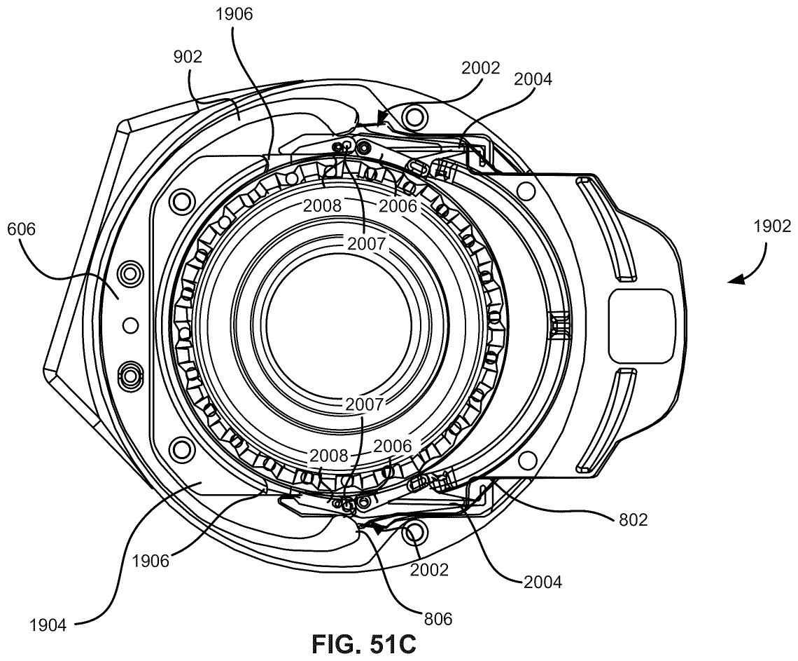

FIG. 51A is a top view of retention related components of the explant plug device 1900. FIG. 51B is a close-up view of lockout-related components of the explant plug device 1900. FIG. 51C is a top view of the retention related components of the explant plug device 1900 in the retracted configuration. In FIG. 51A, a two-part cam mechanism 2002 is shown in contact with the stop surfaces 1906 when the explant plug device 1900 is not properly seated onto the ventricular cuff 120. As illustrated in FIG. 51B, the two-part cam mechanism 2002 is mounted to the retention clip 1902 via machined posts 2009, 2011 on the underside of the retention clip 1902. A spring 2004 contacts a first part 2006 of the two-part cam mechanism 2002 to bias the orientation of the first part 2006 relative to the retention clip 1902. The angle of a second part 2008 of the two-part cam mechanism 2002 is controlled by the orientation of the first part 2006 relative to the retention clip 1902. The spring 2004 biases the first part 2006 inward toward the center of the base member 1904, thereby pushing the moveable post 2007 up and away from the center of the base member 1904. When the explant plug device 1900 is not properly seated onto the ventricular cuff 120, the cuff lock assembly 1902 and the two-part cam mechanism 2002 travel together in this orientation and eventually, the stop surface 1906 abuts the second part 2008 of the two-part cam mechanism 2002 as shown in FIG. 51A and further detailed in the enlarged view shown in FIG. 51B. The interaction between the two-part cam mechanism 2002 and the stop surface 1906 creates the lockout configuration where the retention clip 1902 is prevented from advancing to the locked (retention) configuration.

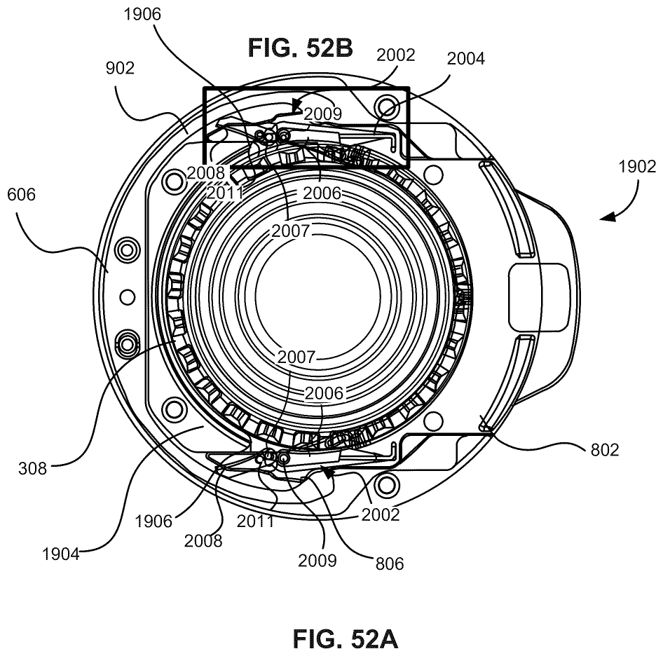

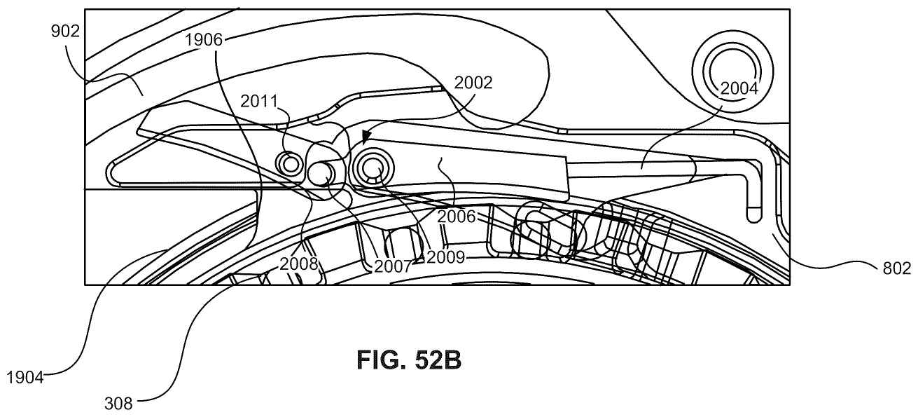

FIG. 52A is a top view of the retention related components of the explant plug device 1900. FIG. 52B is a close-up view of the lockout-related components of the explant plug device 1900 in the locked (retention) configuration. As shown in FIG. 52A, when the explant plug device 1900 is properly seated onto the ventricular cuff 120, the circumferential flange 308 of the ventricular cuff 120 interacts with the two-part cam mechanism 2002 and the spring 2004 is pushed from the extended position to the flexed position. As the retention clip 1902 advances, the cuff flange 308 pushes the first part 2006 of the two-part cam mechanism 2002 against the spring 2004, causing the spring 2004 to flex away from the center of the base member 1904. The springs 2004 can be made of various materials and dimensions for fine tuning the force applied to the first part 2006, in a manner that would be appreciated by one having ordinary skill in the art upon reading the present disclosure. The springs 2004 can have geometries other than those shown in FIG. 52A and FIG. 52B. The cuff flange 308 also causes the first part 2006 to rotate about a first fixed post 2009 in the cuff lock assembly 1902. The moveable post 2007 is directed towards the center of the base member 1904, which causes the second part 2008 to rotate about a second fixed post 2011 so that the free end is directed lateral to the stop surface 1906 of the base member 1904, bypassing it as the retention clip 1902 advances. The retention clip 1902 is able to advance to the locked position having the flexible arms 902 of the spring arm member 606 engage and secure the ridges 806 of the retention clip arms 802.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. For example, various design implementations of the cuff lock assembly of the present invention are shown and described herein. Although each of the designs include a spring arm member for locking the cuff lock assembly in place, configurations without a spring arm member or having a different spring arm member than that shown and described herein are possible. Alternate embodiments can be configured that do not include the spring arm member and/or use alternate mechanisms for securing the cuff lock assembly in the engaged position.

Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications and patents, cited herein are hereby incorporated by reference to the same extent as if each reference were individually and specifically indicated to be incorporated by reference and were set forth in its entirety herein.

1. An explant plug device mountable to a ventricular cuff in fluid communication with a ventricle to block blood from flowing out of the ventricle through a blood flow channel of the ventricular cuff, the explant plug device comprising:
a seal assembly configured for insertion into the blood flow channel and blocking blood flow through the blood flow channel; and
a retention assembly comprising one or more retention members and a spring assembly coupled with the one or more retention members, wherein the retention assembly is reconfigurable between an insertion configuration and a retention configuration, wherein each of the one or more retention members in the insertion configuration is positioned to accommodate insertion of the retention assembly into the blood flow channel of the ventricular cuff, and wherein the spring assembly in the retention configuration is configured to hold each of the one or more retention members in engagement with an inner surface of the ventricular cuff that defines the blood flow channel.

2. The explant plug device of claim 1, wherein:
the spring assembly comprises one or more preloaded springs; and
the preloaded springs are configured to hold each of the one or more retention members in engagement with the inner surface of the ventricular cuff in the retention configuration.

3. The explant plug device of claim 2, wherein:
the one or more retention members comprise a first retention member and a second retention member;
the first retention member is configured for engaging a first annular portion of the inner surface in the retention configuration;
the second retention member is configured for engaging a second annular portion of the inner surface in the retention configuration; and
the second annular portion of the inner surface is disposed opposite to the first annular portion of the inner surface.

4. The explant plug device of claim 1, wherein:
the retention assembly comprises a frame member to which the seal assembly is attached; and
each of the one or more retention members is coupled with the frame member to accommodate movement of the retention member relative to the frame member.

5-7. (canceled)

8. The explant plug device of claim 1, wherein the seal assembly is configured to extend into the ventricle.

9. The explant plug device of claim 1, wherein:
the seal assembly comprises a base assembly, a ventricle opening member, and an adjustment assembly;
the base assembly is mounted to the retention assembly and configured for blocking blood flow through the blood flow channel;
the ventricle opening member is configured for filling an opening in a ventricular wall to which the ventricular cuff is attached;
the ventricle opening member is coupled to the base assembly via the adjustment assembly; and
the adjustment assembly is operable to adjust a distance between the ventricle opening member and the base assembly.

10-40. (canceled)

41. An explant plug device mountable to a ventricular cuff in fluid communication with a ventricle to block blood from flowing out of the ventricle through the ventricular cuff, wherein the ventricular cuff comprises an outwardly facing annular flange, an outwardly facing annular groove, and a blood flow channel for an inlet cannula of a blood pump, the explant plug device comprising:
a blood flow blocking component configured to be mounted to the ventricular cuff to block flow of blood out of the ventricle via the ventricular cuff; and
a retention clip coupled with the blood flow blocking component, wherein the retention clip is repositionable relative to the blood flow blocking component from a retracted position that accommodates mounting of the blood flow blocking component to the ventricular cuff and a retention position that engages the outwardly facing annular flange to retain the blood flow blocking component to the ventricular cuff.

42. The explant plug device of claim 41, wherein the retention clip comprises retention clip arms that extend within retention clip arm passages of the blood flow blocking component and into the outwardly facing annular groove when the retention clip is in the retention position.

43. The explant plug device of claim 42, wherein, when the blood flow blocking component is not mounted onto the ventricular cuff, the retention clip is blocked from being repositioned to the retention position from the retracted position.

44. The explant plug device of claim 43, further comprising a lockout component pivotally coupled with the retention clip, wherein:
the lockout component is held in a blocking orientation relative to the retention clip in which the lockout component blocks the retention clip from being repositioned to the retention position from the retracted position when the blood flow blocking component is not mounted onto the ventricular cuff; and
mounting of the blood flow blocking component to the ventricular cuff reorients the lockout component relative to the retention clip from the blocking orientation to a non-blocking orientation that accommodates repositioning of the retention clip to the retention position from the retracted position.

45. The explant plug device of claim 44, wherein the retention clip comprises a spring arm that engages the lockout component to hold the lockout component in the blocking orientation when the blood flow blocking component is not mounted onto the ventricular cuff.

46. The explant plug device of claim 44, further comprising a recess, wherein:
the recess is configured to accommodate a protruding portion of the lockout component; and
the recess is configured to engage the protruding portion of the lockout component to block the retention clip from being repositioned to the retention position from the retracted position when the lockout component is in the blocking orientation.

47. The explant plug device of claim 43, further comprising a stop component, wherein:
the stop component is biased to a blocking position when the blood flow blocking component is not mounted to the ventricular cuff;
the stop component in the blocking position is configured to engage the retention clip to block repositioning of the retention clip to the retention position from the retracted position; and
mounting of the blood flow blocking component to the ventricular cuff repositions the stop component from the blocking position to a non-blocking position that accommodates repositioning of the retention clip to the retention position from the retracted position.

48. The explant plug device of claim 47, further comprising a stop component spring that biases the stop component to the blocking position when the blood flow blocking component is not mounted to the ventricular cuff, and wherein the stop component is configured to be engaged by the ventricular cuff during mounting of the blood flow blocking component to the ventricular cuff to displace the stop component from the blocking position to the non-blocking position.

49. The explant plug device of claim 43, further comprising a two-part cam mechanism mounted to the retention clip, wherein:
the two-part cam mechanism is biased to a blocking configuration when the blood flow blocking component is not mounted to the ventricular cuff;
the two-part cam mechanism in the blocking configuration is configured to block repositioning of the retention clip to the retention position from the retracted position; and
mounting of the blood flow blocking component to the ventricular cuff reconfigures the two-part cam mechanism from the blocking configuration to a non-blocking configuration that accommodates repositioning of the retention clip to the retention position from the retracted position.

50. The explant plug device of claim 49, wherein the retention clip comprises a spring that engages the two-part cam mechanism to hold the two-part cam mechanism in the blocking configuration when the blood flow blocking component is not mounted onto the ventricular cuff.

51. The explant plug device of claim 43, further comprising a spring arm member coupled with the blood flow blocking component, wherein the spring arm member comprises flexible arms configured to engage the retention clip arms in the retention position to inhibit repositioning of the retention clip relative to the blood flow blocking component.

52. The explant plug device of claim 51, wherein:
each of the flexible arms engages with a distal end portion of a respective one of the retention clip arms; and
each of the distal end portions of the retention clip arms is shaped to inhibit repositioning of the retention clip from the retention position.

53. The explant plug device of claim 43, wherein:
the blood flow channel of the ventricular cuff has a longitudinal axis; and
the retention clip is constrained to be moved, relative to the ventricular cuff, from the retracted position to the retention position perpendicular to the longitudinal axis.

54. (canceled)

55. The explant plug device of claim 41, wherein:
the retention clip includes a visual indicator,
the visual indicator is disposed external to the blood flow blocking component when the retention clip is not in the retention position; and
the visual indicator is disposed within the blood flow blocking component when the retention clip is in the retention position.
